SEQUENCE LISTING (100 sequences)

SEQ ID NO: 1 - DNA, 873 bp, Human
atcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccagggg

SEQ ID NO: 2 - DNA, 519 bp, Human
atcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaag

SEQ ID NO: 3 - protein, 291 aa, Human
ILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQG

SEQ ID NO: 4 - protein, 174 aa, Human
IPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKI

SEQ ID NO: 5 - DNA, 45 bp, Human
ctgcactggctgatgagtgtgtacgtcgtcgagctgctcaggtct

SEQ ID NO: 6 - DNA, 45 bp, Human
ctctttttctaccggaagagtgtctggagcaagttgcaaagcatt

SEQ ID NO: 7 - DNA, 45 bp, Human
acgtccagactccgcttcatccccaagcctgacgggctgcggccg

SEQ ID NO: 8 - DNA, 45 bp, Human
cgccccggcctcctgggcgcctctgtgctgggcctggacgatatc

SEQ ID NO: 9 - DNA, 45 bp, Human
tttgcggggattcggcgggacgggctgctcctgcgtttggtggat

SEQ ID NO: 10 - DNA, 45 bp, Human
tatggctgcgtggtgaacttgcggaagacagtggtgaacttccct

SEQ ID NO: 11 - DNA, 45 bp, Human
ggcacggcttttgttcagatgccggcccacggcctattcccctgg

SEQ ID NO: 12 - DNA, 45 bp, Human
tggtgcggcctgctgctggatacccggaccctggaggtgcagagc

SEQ ID NO: 13 - DNA, 45 bp, Human
gcgaaaaccttcctcaggaccctggtccgaggtgtccctgagtat

SEQ ID NO: 14 - DNA, 60 bp, Human
cggccgattgtgaacatggactacgtcgtgggagccagaacgttccgcagagaaaagagg

SEQ ID NO: 15 - DNA, 42 bp, Human
ctgtactttgtcaaggtggatgtgacgggcgcgtacgacacc

SEQ ID NO: 16 - DNA, 45 bp, Human
cggacctccatcagagccagtctcaccttcaaccgcggcttcaag

SEQ ID NO: 17 - protein, 15 aa, Human
LHWLMSVYVVELLRS

SEQ ID NO: 18 - protein, 15 aa, Human
LFFYRKSVWSKLQSI

SEQ ID NO: 19 - protein, 15 aa, Human
TSRLRFIPKPDGLRP

SEQ ID NO: 20 - protein, 15 aa, Human
RPGLLGASVLGLDDI

SEQ ID NO: 21 - protein, 15 aa, Human
FAGIRRDGLLLRLVD

SEQ ID NO: 22 - protein, 15 aa, Human
YGCVVNLRKTVVNFP

SEQ ID NO: 23 - protein, 15 aa, Human
GTAFVQMPAHGLFPW

SEQ ID NO: 24 - protein, 15 aa, Human
WCGLLLDTRTLEVQS

SEQ ID NO: 25 - protein, 15 aa, Human
AKTFLRTLVRGVPEY

SEQ ID NO: 26 - protein, 20 aa, Human
RPIVNMDYVVGARTFRREKR

SEQ ID NO: 27 - protein, 14 aa, Human
LYFVKVDVTGAYDT

SEQ ID NO: 28 - protein, 15 aa, Human
CHSLFLDLQVNSLQT

SEQ ID NO: 29 - protein, 15 aa, Human
AKFLHWLMSVYVVEL

SEQ ID NO: 30 - protein, 15 aa, Human
LMSVYVVELLRSFFY

SEQ ID NO: 31 - protein, 15 aa, Human
MSVYVVELLRSFFYV

SEQ ID NO: 32 - protein, 15 aa, Human
YVVELLRSFFYVTET

SEQ ID NO: 33 - protein, 15 aa, Human
VELLRSFFYVTETTF

SEQ ID NO: 34 - protein, 15 aa, Human
SFFYVTETTFQKNRL

SEQ ID NO: 35 - protein, 15 aa, Human
KNRLFFYRKSVWSKL

SEQ ID NO: 36 - protein, 15 aa, Human
KSVWSKLQSIGIRQH

SEQ ID NO: 37 - protein, 15 aa, Human
WSKLQSIGIRQHLKR

SEQ ID NO: 38 - protein, 15 aa, Human
QSIGIRQHLKRVQLR

SEQ ID NO: 39 - protein, 15 aa, Human
SIGIRQHLKRVQLRE

SEQ ID NO: 40 - protein, 15 aa, Human
RQHLKRVQLRELSEA

SEQ ID NO: 41 - protein, 15 aa, Human
RPALLTSRLRFIPKP

SEQ ID NO: 42 - protein, 15 aa, Human
PDGLRPIVNMDYVVG

SEQ ID NO: 43 - protein, 15 aa, Human
LRPIVNMDYVVGART

SEQ ID NO: 44 - protein, 15 aa, Human
RPIVNMDYVVGARTF

SEQ ID NO: 45 - protein, 15 aa, Human
NMDYVVGARTFRREK

SEQ ID NO: 46 - protein, 15 aa, Human
ARTFRREKRAERLTS

SEQ ID NO: 47 - protein, 15 aa, Human
AERLTSRVKALFSVL

SEQ ID NO: 48 - protein, 15 aa, Human
VKALFSVLNYERARR

SEQ ID NO: 49 - protein, 15 aa, Human
LFSVLNYERARRPGL

SEQ ID NO: 50 - protein, 15 aa, Human
ASVLGLDDIHRAWRT

SEQ ID NO: 51 - protein, 15 aa, Human
HRAWRTFVLRVRAQD

SEQ ID NO: 52 - protein, 15 aa, Human
WRTFVLRVRAQDPPP

SEQ ID NO: 53 - protein, 15 aa, Human
VLRVRAQDPPPELYF

SEQ ID NO: 54 - protein, 15 aa, Human
ELYFVKVDVTGAYDT

SEQ ID NO: 55 - protein, 15 aa, Human
TYCVRRYAVVQKAAH

SEQ ID NO: 56 - protein, 15 aa, Human
VRRYAVVQKAAHGHV

SEQ ID NO: 57 - protein, 15 aa, Human
HGHVRKAFKSHVSTL

SEQ ID NO: 58 - protein, 15 aa, Human
RKAFKSHVSTLTDLQ

SEQ ID NO: 59 - protein, 15 aa, Human
LTDLQPYMRQFVAHL

SEQ ID NO: 60 - protein, 15 aa, Human
QPYMRQFVAHLQETS

SEQ ID NO: 61 - protein, 15 aa, Human
TSPLRDAVVIEQSSS

SEQ ID NO: 62 - protein, 15 aa, Human
RDAVVIEQSSSLNEA

SEQ ID NO: 63 - protein, 15 aa, Human
SGLFDVFLRFMCHHA

SEQ ID NO: 64 - protein, 15 aa, Human
LFDVFLRFMCHHAVR

SEQ ID NO: 65 - protein, 18 aa, Human
FDVFLRFMCHHAVRIRGK

SEQ ID NO: 66 - protein, 15 aa, Human
HHAVRIRGKSYVQCQ

SEQ ID NO: 67 - protein, 15 aa, Human
GKSYVQCQGIPQGSI

SEQ ID NO: 68 - protein, 17 aa, Human
RDGLLLRLVDDFLLVTP

SEQ ID NO: 69 - protein, 20 aa, Human
DFLLVTPHLTHAKTFLRTLV

SEQ ID NO: 70 - protein, 15 aa, Human
KTFLRTLVRGVPEYG

SEQ ID NO: 71 - protein, 20 aa, Human
AHGLFPWCGLLLDTRTLEVQ

SEQ ID NO: 72 - protein, 19 aa, Human
TLEVQSDYSSYARTSIRAS

SEQ ID NO: 73 - protein, 15 aa, Human
QSDYSSYARTSIRAS

SEQ ID NO: 74 - protein, 20 aa, Human
RTSIRASLTFNRGFKAGRNM

SEQ ID NO: 75 - protein, 18 aa, Human
RRKLFGVLRLKCHSLFLD

SEQ ID NO: 76 - protein, 20 aa, Human
HSLFLDLQVNSLQTVCTNIY

SEQ ID NO: 77 - protein, 15 aa, Human
RTSIRASLTFNRGFK

SEQ ID NO: 78 - protein, 15 aa, Epstein Barr Virus
LSTDVGSCTLVCPLH

SEQ ID NO: 79 - protein, 15 aa, Epstein Barr Virus
AYFMVFLQTHIFAEV

SEQ ID NO: 80 - protein, 20 aa, Human
RRKLFGVLRLKCHSLFLDLQ

SEQ ID NO: 81 - protein, 500 aa, Human
EHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFF

SEQ ID NO: 82 - protein, 9 aa, Human
LMSVYVVEL

SEQ ID NO: 83 - protein, 9 aa, Human
YMRQFVAHL

SEQ ID NO: 84 - protein, 9 aa, Human
LLLRLVDDF

SEQ ID NO: 85 - protein, 9 aa, Human
FLRTLVRGV

SEQ ID NO: 86 - protein, 11 aa, Human
GLLLDTRTLEV

SEQ ID NO: 87 - protein, 9 aa, Human
ASLTFNRGF

SEQ ID NO: 88 - protein, 9 aa, Human
FLDLQVNSL

SEQ ID NO: 89 - protein, 15 aa, Human
LYFVKVDVTGAYDTI

SEQ ID NO: 90 - protein, 19 aa, Human
LFDVFLRFMCHHAVRIRGK

SEQ ID NO: 91 - protein, 15 aa, Human
FAGIRRDGLLLRLVD

SEQ ID NO: 92 - protein, 15 aa, Human
WCGLLLDTRTLEVQS

SEQ ID NO: 93 - protein, 15 aa, Human
RTSIRASLTFNRGFK

SEQ ID NO: 94 - protein, 19 aa, Human
RRKLFGVLRLKCHSLFLDL

SEQ ID NO: 95 - DNA, 45 bp, Human
ctgtactttgtcaaggtggatgtgacgggcgcgtacgacaccatc

SEQ ID NO: 96 - DNA, 57 bp, Human
ctcttcgacgtcttcctacgcttcatgtgccaccacgccgtgcgcatcaggggcaag

SEQ ID NO: 97 - DNA, 45 bp, Human; identical to SEQ ID NO: 9

SEQ ID NO: 98 - DNA, 45 bp, Human; identical to SEQ ID NO: 12

SEQ ID NO: 99 - DNA, 45 bp, Human; identical to SEQ ID NO: 16

SEQ ID NO: 100 - DNA, 57 bp, Human
cgtcgcaaactctttggggtcttgcggctgaagtgtcacagcctgtttctggatttg

(19) United States
(12) Patent Application Publication
(10) Pub. No.: US 2003/0143228 A1
(43) Pub. Date: Jul. 31, 2003

(54) Human telomerase reverse transcriptase as a class-II restricted tumor-associated antigen

(75) Inventors: Si-Yi Chen, Pearland, TX (US); Roland Schroers, North Hardenberg (DE)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX

(21) Appl. No.: 10/282,960

(22) Filed: Oct. 29, 2002

Related U.S. Application Data
(60) Provisional application No. 60/345,012, filed on Oct. 29, 2001.

(51) Int. Cl. A61K 39/395; C07H 21/04; C07K 16/28; C12P 21/02; C12N 5/06; C07K 14/74

(52) U.S. Cl. 424/144.1; 530/388.22; 435/69.1; 435/320.1; 536/23.53; 435/334

(57) ABSTRACT

The present invention relates to the identification of MHC-I and MHC-II hTRT restricted epitopes and the use of these identified epitopes to elicit an immune response against the epitope. More particularly, the identified epitopes are administered to a subject to treat hyperproliferative diseases.

This application claims priority to U.S. Provisional Application Serial No. 60/345,012, which was filed on Oct. 29, 2001.

This invention was made using funds obtained from the U.S. Government and the U.S. Government may therefore have certain rights in the invention.

### BACKGROUND OF THE INVENTION

A. Field of Invention

The present invention relates to the identification of hTRT restricted epitopes and the use of these identified epitopes to elicit an immune response against the epitope. More particularly, the present invention uses the identified epitopes to treat hyperproliferative diseases.

B. Description of Related Art

CD4+ helper T-cells (Th), which recognize major histocompatibility complex (MHC) class II-restricted tumor-associated antigens (TAA), play critical roles in initiating, regulating, and maintaining antitumor immune responses (Pardoll et al., 1998; Rosenberg, 1999). Dissection of immune cell interactions has revealed the requirement for epitope linkage between class II-restricted and class I-restricted epitopes for the induction of potent antitumor responses (Bennett et al., Schoenberger et al., 1998). Despite the importance of CD4+ Th (Pardoll et al., 1998; Rosenberg, 1999), only a few MHC class II-restricted TAA are available for the development of tumor vaccines/immunotherapy (Topalian et al., 1994; Chaux et al., 1999; Manici et al., 1999; Wang et al., 1999). In contrast to the successful identification of MHC class I-restricted TAA recognized by CD8+ cytotoxic T-cells (CTL) (van der Bruggen et al., 1991), class II-restricted TAA are difficult to detect (Pardoll et al., 1998; Rosenberg, 1999).

Current approaches to identifying class II-restricted TAA rely on scarce primed tumor-specific CD4+ T-cells (Topalian et al., 1994; Wang et al., 1999), which severely limits their application. For instance, a biochemical approach, based upon the detection of tumor-specific CD4+ T-cell responses to peptides eluted from class II-positive tumor cells (Topalian et al., 1994), is limited by the scarcity of primed tumor-specific CD4+ T-cells and the difficulty of purifying minuscule amounts of class II-restricted peptides from tumor cells. An improved approach, reported recently (Wang et al., 1999), is to transfect DNA libraries into non-antigen presenting cell (APC) genetically modified to express appropriate MHC molecules, followed by identification of transfectants capable of stimulating tumor-specific human CD4+ T-cells. Although used successfully to identify a mutated antigen, this approach cannot prime T-cells and is still limited by the scarcity of primed tumor-specific CD4+ T-cells.

The immunogenetic screening approach developed in the present invention has the unique ability to prime human CD4+ Th cells in vitro and can be applied to the identification of any class II-restricted antigens associated with tumors, infectious diseases, autoimmune diseases, or allergic diseases.

### BRIEF SUMMARY OF THE INVENTION

The present invention is directed to polynucleotide and amino acid sequences of human telomerase reverse transcriptase MHC-I and MHC-II restricted epitopes. It is envisioned that these epitopes are used to elicit an immune response against the epitope resulting in a treatment for hyperproliferative diseases. These epitopes were identified using a retrogen strategy. Yet further, the present invention comprises the treatment of hyperproliferative diseases using the identified epitopes. The treatment of such a hyperproliferative disease involves the administration of the polynucleotide and/or amino acid sequences of the present invention. It is contemplated that the polynucleotide sequences of the present invention are inserted into an expression vector that is administered to a subject. Yet further, the expression vector is transduced into antigen presenting or tumor cells, which are then administered to a subject. It is also contemplated that the amino acid sequences of the present invention can be administered to the subject or pulsed into antigen presenting cells or tumor cells.

An embodiment of the present invention is an isolated polynucleotide sequence comprising the nucleic acid sequence of SEQ.ID.NO.1 or SEQ.ID.NO.2. In specific embodiments, the nucleic acid sequences of SEQ.ID.NO.1 or SEQ.ID.NO.2 are inserted into an an expression vector. Yet further, it is envisioned that the expression vectors are used to produce transformed cells. Still further, it is contemplated that the expression vectors are administered to a subject to elicit an immune response. For example, a method of the present invention is a method of eliciting an immune response directed against an antigen, comprising the step of administering to a subject an expression vector of the present invention.

Another embodiment of the present invention is an isolated polypeptide comprising the amino acid sequence of SEQ.ID.NO.3, SEQ.ID.NO.4 or SEQ.ID.NO.59. Specifically, the amino acid sequences of SEQ.ID.NO.3 and SEQ.ID.NO.4 comprises epitopes that binds to MHC-I and MHC-II. The amino acid sequence of SEQ.ID.NO.59 comprises epitopes that binds to MHC-II. Yet further, the polypeptide compositions are used to ellict and immune response.

Yet further, another embodiment is an expression vector comprising a polynucleotide encoding signal sequence, a polynucleotide encoding at least one epitope of human telomerase reverse transcriptase (hTRT), a polynucleotide encoding a cell binding element and a polynucleotide encoding a dendritic cell receptor, all operatively linked. Specifically, the epitope induces a CD4+ T-cell response or induces a CD4+ T-cell and a CD8+ T-cell response in a mammal. In particular embodiments, the epitope of hTRT is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15 and SEQ.ID.NO.16. It is contemplated that the expression vector is used to produce transformed cells.

Still further, another embodiment is an expression vector comprising a polynucleotide encoding signal sequence, a first polynucleotide sequence encoding at least one epitope of hTRT, a second sequence polynucleotide encoding at least one epitope of hTRT, a polynucleotide sequence encoding a cell binding element and a polynucleotide sequence encoding a dendritic cell receptor, all operatively linked. In specific embodiments, the first and second polynucleotide sequences encoding at least one epitope of hTRT are separated by an internal ribosome entry site or are in tandem and under the control of one promoter. Yet further, the first polynucleotide sequence encoding at least one epitope of hTRT encodes an epitope that binds to a MHC-II receptor and/or a MHC-I receptor. The second polynucleotide sequence encoding at least one epitope of hTRT encodes an epitope that binds to a MHC-II receptor and/or a MHC-I receptor. More particularly, the polynucleotide sequence is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15 and SEQ.ID.NO.16. It is contemplated that the expression vector is used to produce transformed cells.

Another embodiment of the present invention is an expression vector comprising a two transgenes, wherein the first and second transgene comprises a promoter polynucleotide sequence, a polynucleotide encoding signal sequence, a polynucleotide sequence encoding at least one epitope of hTRT, a polynucleotide sequence encoding a cell binding element, and a polynucleotide sequence encoding a dendritic cell receptor, all operatively linked. Specifically, the promoter polynucleotide sequence is the same or is different for the first transgene and second transgene. Yet further, the polynucleotide sequence is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100. It is contemplated that the expression vector is used to produce transformed cells. Still further, a method of the present invention is a method of eliciting an immune response directed against an antigen, comprising the step of administering to a subject an expression vector, transformed cell and/or cell lysate of the transformed cell of the present invention.

Another specific embodiment of the present invention is a method of eliciting an immune response directed against an antigen comprising the step of administering to a subject a peptide selected from the group consisting of SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62, SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

Still further, a method of the present invention is a method of eliciting an immune response directed against an antigen, comprising the step of administering to a subject an expression vector, transformed cell and/or cell lysate of the transformed cell of the present invention.

Another embodiment of the present invention is a method of treating a hyperproliferative disease comprising the step of administering transduced antigen presenting cells to a subject via a parenteral route. The hyperproliferative disease is further defined as cancer. Yet further, the cancer is selected from the group consisting of lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In specific embodiments, the antigen presenting cells are autologous or allogeneic to the subject. The antigen presenting cells are pulsed with an expression vector comprising a polynucleotide sequence of hTRT, wherein said polynucleotide sequence of is selected from the group consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100. More particularly, the antigen presenting cells are pulsed with a peptide selected from the group consisting of SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62, SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

Another embodiment is a method of treating a hyperproliferative disease comprising the step of administering to a subject an expression vector with a pharmaceutical acceptable carrier, wherein said expression vector comprises a polynucleotide promoter sequence, a polynucleotide encoding a signal sequence, a polynucleotide encoding an at least one epitope of hTRT, and a polynucleotide encoding a cell binding element and a polynucleotide sequence encoding a dendritic cell receptor, all operatively linked. Particularly, the epitope of hTRT is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.142, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100.

Still further, another embodiment is a method of treating a hyperproliferative disease comprising administering to a subject a hTRT specific peptide with a pharmaceutical acceptable carrier, wherein said peptide binds to a MHC-II receptor. More specifically, thed hTRT peptide is selected from the group of consisting of SEQ.ID.NO. 3, SEQ.ID.NO. 4, SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62, SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

Another embodiment of the present invention is a method of treating a hyperproliferative disease comprising administering to a subject a hTRT specific peptide with a pharmaceutical acceptable carrier, wherein said peptide binds to a MHC-I and MHC-II receptor. Specifically, the hTRT peptide is selected from the group of consisting of SEQ.ID.NO. 3, SEQ.ID.NO. 4, SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62, SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

Still further, a method of the present invention is a method of treating a hyperproliferative disease comprising the step of administering to a subject an expression vector, transformed cell and/or cell lysate of the transformed cell of the present invention.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF SUMMARY OF THE DRAWINGS

For a more complete understanding of the present invention, reference is now made to the following descriptions taken in conjunction with the accompanying drawings.

FIG. 1 shows in vitro priming of naïve human CD4+ T-cells by transduced DC.

FIG. 2A-FIG. 2C show the identification of hTRT as a class II-restricted tumor antigen. FIG. 2A shows the identification of positive clones from tumor retrogen library. FIG. 2B shows the antibody blocking experiment. FIG. 2C shows the amino acid sequences of clones 8 (SEQ.ID.NO. 3) and 35 (SEQ.ID.NO. 4). Amino acid sequences translated from the DNA sequences of positive clones 8 and 35, with hTRT alignment (SEQ.ID.NO.81), are shown. The MHC class II-restricted epitopes in clones 8 and 35 that were positively identified later are underlined.

FIG. 3A-FIG. 3L show the identification of class II-restricted epitopes in hTRT. FIG. 3A shows the proliferative T-cell responses to hTRT-derived peptide T573 donor B-16. FIG. 3B shows the proliferative T-cell responses to hTRT-derived peptide T573 donor B-22. FIG. 3C shows the proliferative T-cell responses to hTRT-derived peptide T672 donor B-22. FIG. 3D shows the proliferative T-cell responses to hTRT-derived peptide T672 donor B-24. FIG. 3E shows the proliferative T-cell responses to hTRT-derived peptide T672 donor B-05. FIG. 3F shows the proliferative T-cell responses to hTRT-derived peptide T880 donor B-05. FIG. 3G shows the proliferative T-cell responses to hTRT-derived peptide T880 donor B-14. FIG. 3H shows the proliferative T-cell responses to hTRT-derived peptide T880 donor B-15. FIG. 31 shows the proliferative T-cell responses to hTRT-derived peptide T916 donor B-24. FIG. 3J shows the proliferative T-cell responses to hTRT-derived peptide T916 donor B-22. FIG. 3K shows the proliferative T-cell responses to hTRT-derived peptide T916 donor B-15. FIG. 3L shows the proliferative T-cell responses to hTRT-derived peptide T916 donor B-05.

FIG. 4A and FIG. 4B show [³H]-thymidine incorporations of the primed T-cells were measured after re-stimulation with autologous PBMCs with (black bar) or without (white bar) corresponding peptides. FIG. 4B shows the generation of T-cell clones. FIG. 4C shows the specificity of T-cell responses.

FIG. 5 shows the specificity of T-cell responses for the hTRT672-positive T-cell line.

FIG. 6 shows the specificity of T-cell responses for the hTRT631-positive T-cell line.

FIG. 7 shows the peptide titration of T-cell responses for hTRT916 and hTRT672 CD4+ T-cell clones.

FIG. 8 shows the peptide titration of T-cell responses for hTRT631 CD4+ T-cell clones.

FIG. 9A and FIG. 9B show the flow cytometric assay of T-cell clones.

FIG. 10 shows the T-cell response to natively processed hTRT672.

FIG. 11A and FIG. 11B show the CD4+ T-cell responses to different tumors.

FIG. 12A-FIG. 12J show the proliferative T-cell responses to hTRT-derived peptides. FIG. 12A shows the T-cell response for hTRT631. FIG. 12B shows the T-cell response for hTRT706. FIG. 12C shows the T-cell response for hTRT854. FIG. 12D shows the T-cell response for hTRT894. FIG. 12E shows the T-cell response for hTRT930. FIG. 12F shows the T-cell response for hTRT951. FIG. 12G shows the T-cell response for hTRT766. FIG. 12H shows the T-cell response for hTRT787. FIG. 121 shows the T-cell response for hTRT805. FIG. 12J shows the T-cell response for hTRT971.

FIG. 13A-FIG. 13F show the specificity and MHC-restriction of T-cell responses. FIG. 13A shows the response for hTRT631. FIG. 13B shows the response for hTRT706. FIG. 13C shows the response for hTRT766. FIG. 13D shows the response for hTRT787. FIG. 13E shows the response for hTRT805. FIG. 13F shows the response for hTRT894.

FIG. 14A-FIG. 14F shows the FACS analysis of T-cell clones. FIG. 14A shows FACS analysis for hTRT631. FIG. 14B shows FACS analysis for hTRT706. FIG. 14C shows FACS analysis for hTRT766. FIG. 14D shows FACS analysis for hTRT787. FIG. 14E shows FACS analysis for hTRT805. FIG. 14F shows FACS analysis for hTRT894.

FIG. 15A-FIG. 15F shows the peptide titration experiments. FIG. 15A shows the peptide concentration for hTRT631. FIG. 15B shows peptide concentration for hTRT706. FIG. 15C shows peptide concentration for hTRT766. FIG. 154D shows peptide concentration for hTRT787. FIG. 15E shows peptide concentration for hTRT805. FIG. 15F shows peptide concentration for hTRT894.

FIG. 16 shows analysis of recombinant hTRT protein.

FIG. 17 shows tesponses of T-cell clone hTRT766 to natively processed hTRT protein.

FIG. 18A and FIG. 18B show the presentation of of hTRT peptides. FIG. 18A shows the presentation of hTRT672 by different HLA-DR alleles. FIG. 18B shows the presentation of hTRT766 by different HLA-DR alleles.

FIG. 19 shows the peptide-specific Th response induced by immunization with hTRT766.

FIG. 20 shows the Th responses to antigenic peptides derived from hTRT proteins and hTRT-positive tumor lysates.

### DETAILED DESCRIPTION OF THE INVENTION

It is readily apparent to one skilled in the art that various embodiments and modifications can be made to the invention disclosed in this Application without departing from the scope and spirit of the invention.

As used herein, the use of the word “a” or “an” when used in conjunction with the term “comprising” in the claims and/or the specification may mean “one,” but it is also consistent with the meaning of “one or more,” “at least one,” and “one or more than one.”

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention exists in a variety of forms including, for example, polyclonal antibodies, monoclonal antibodies, Fv, Fab and F(ab)₂, as well as single chain antibodies and humanized antibodies (Harlow et al., 1988; Houston et al., 1988; Bird et al., 1988).

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates. Exemplary organisms include but are not limited to, Helicobacters, Campylobacters, Clostridia, Corynebacterium diphtheriae, Bordetella pertussis, influenza virus, parainfluenza viruses, respiratory syncytial virus, Borrelia burgdorfei, Plasmodium, herpes simplex viruses, human immunodeficiency virus, papillomavirus, Vibrio cholera, E. coli, measles virus, rotavirus, shigella, Salmonella typhi, Neisseria gonorrhea. Therefore, a skilled artisan realizes that any macromolecule, including virtually all proteins or peptides, can serve as antigens. Furthermore, antigens can be derived from recombinant or genomic DNA. A skilled artisan realizes that any DNA, which contains nucleotide sequences or partial nucleotide sequences of a pathogenic genome or a gene or a fragment of a gene for a protein that elicits an immune response results in synthesis of an antigen. Furthermore, one skilled in the art realizes that the present invention is not limited to the use of the entire polynucleotide sequence of a gene or genome. It is readily inherent that the present invention includes, but is not limited to, the use of partial polynucleotide sequences of more than one gene or genome and that these polynucleotide sequences are arranged in various combinations to elicit the desired immune response.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is preferred, such as where the non-coding regions are required for optimal expression or where non-coding regions such as introns are to be targeted in an antisense strategy.

The term “cell binding element” as used herein is defined as a portion of a protein, which is capable of binding to a receptor on a cell membrane.

The term “DNA” as used herein is defined as deoxyribonucleic acid.

The term “dendritic cell” or “DC” as used herein is defined as an example of an antigen presenting cell derived from bone marrow. Dendritic cells have a branched or dendritic morphology and are the most potent stimulations of T-cell response.

The term “dendritic cell receptor” as used herein is defined as a cell surface protein on a dendritic cell that recognize and bind specific proteins, for example intracellular adhesion molecules (ICAM). One specific ICAM is ICAM-3.

The term “epitope” as used herein is defined as small chemical groups on the antigen molecule that can elicit and react with an antibody. An antigen can have one or more epitopes. Most antigens have many epitopes; i.e., they are multivalent. In general, an epitope is about 5 amino acids or sugars in size. One skilled in the art understands that generally the overall three-dimensional structure, rather than the specific linear sequence of the molecule, is the main criterion of antigenic specificity.

The term “expression construct” or “transgene” as used herein is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” can also be used to refer to the expression construct or transgene. One skilled in the art realizes that the present invention utilizes the expression construct or transgene as a therapy to treat hyperproliferative diseases.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors can contain nucleic acid sequences that serve other functions as well and are described infra.

The term “gene” as used herein is defined as a functional protein, polypeptide, of peptide-encoding unit. As will be understood by those in the art, this functional term includes genomic sequences, cDNA sequences, and smaller engineered gene segments that express, or is adapted to express, proteins, polypeptides, domains, peptides, fusion proteins, and mutants.

The term “helper T-cell” as used herein is defined as effector T-cells whose primary function is to promote the activation and functions of other B and T lymphocytes and of macrophages. Most are CD4 T-cells.

The term “heterologous” as used herein is defined as DNA or RNA sequences or proteins that are derived from the different species.

The term “homologous” as used herein is defined as DNA or RNA sequences or proteins that are derived from the same species.

The term “hyperproliferative disease” as used herein refers to any disease or disorder in which the cells proliferate more rapidly than normal tissue growth. Thus, a hyperproliferating cell is a cell that is proliferating more rapidly than normal cells. Hyperproliferative disease is further defined as cancer. The hyperproliferation of cells results in unregulated growth, lack of differentiation, local tissue invasion, and metastasis. Exemplary hyperproliferative diseases include, but are not limited to cancer or immune-mediated diseases.

The term “immune-mediated disease” as used herein refers to chronic inflammatory diseases perpetuated by antibodies and cellular immunity. Immune-mediated diseases include, for example, but not limited to, arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis), vascular diseases (e.g., autoimmune hearing loss, systemic vasculitis, and atherosclerosis), and skin diseases (e.g., dermatomyositis, systemic lupus erthematosus, discoid lupus erthematosus, scleroderma, and vasculitics).

The term “major histocompatibility complex,” or “MHC,” as used herein is defined as a cluster of genes, many of which encode evolutionarily related cell surface proteins involved in antigen presentation, which are among the most important determinants of histocompatability. MHC Class I, or MHC-I, functions mainly in antigen presentation to CD8+ T lymphocytes. MHC Class II, or MHC-II, functions mainly in antigen presentation to CD4+ T lymphocytes.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include with mutations of the polynucleotides, including but not limited to, mutation of the nucleotides, or nucleosides by methods well known in the art.

The term “polypeptide” as used herein is defined as a chain of amino acid residues, usually having a defined sequence. As used herein the term polypeptide includes both “peptides” and “proteins”.

The term “promoter” as used herein is defined as a DNA sequence recognized by the synthetic machinery of the cell, or introduced synthetic machinery, required to initiate the specific transcription of a polynucleotide sequence.

The term “retrogen” as used herein, means a polypeptide having an epitope that is capable of eliciting an immune response in a mammal when expressed and processed as described herein.

The term “retrogen expression vector” as used herein refers to the expression vector comprising at least a polynucleotide sequence encoding a signal sequence, a polynucleotide sequence encoding an antigen and a polynucleotide sequence encoding a cell binding element. It is also contemplated that the retrogen expression vector can include a polynucleotide sequence encoding a dendritic cell receptor.

The term “RNA” as used herein is defined as ribonucleic acid. The term “recombinant DNA” as used herein is defined as DNA produced by joining pieces of DNA from different sources.

The term “recombinant polypeptide” as used herein is defined as a hybrid protein produced by using recombinant DNA methods.

The term “subject” as used herein, is taken to mean any mammalian subject. In a specific embodiment, the methods of the present invention are employed to treat a human subject. Another embodiment includes treating a human subject suffering from a hyperproliferative disease.

The term “T-cell” as used herein is defined as a thymus-derived cell that participates in a variety of cell-mediated immune reactions.

The term “transfected” or “transformed” or “transduced” as used herein refers to a process by which exogenous nucleic acid are transferred or introduced into the host cell. A transformed cell includes the primary subject cell and its progeny.

The phrase “under transcriptional control” or “operatively linked” as used herein means that the promoter is in the correct location and orientation in relation to the polynucleotides to control RNA polymerase initiation and expression of the polynucleotides.

The present invention comprises polynucleotide and amino acid sequences of human telomerase reverse transcriptase MHC-I and MHC-II restricted epitopes. These epitopes were identified using a retrogen strategy. Yet further, the present invention contemplates the treatment of hyperproliferative diseases. The treatment of such a hyperproliferative disease involves the administration of the polynucleotide and/or amino acid sequences of the present invention. It is contemplated that the polynucleotide sequences of the present invention are inserted into an expression vector, which is administered to a subject. Yet further, the expression vector is transduced into antigen presenting or tumor cells, which are then administered to a subject. It is also contemplated that the amino acid sequences of the present invention can be administered to the subject or pulsed into antigen presenting cells or tumor cells.

I. hTRT Peptides

Isolated amino acid sequences for human telomerase reverse transcriptase (hTRT) MHC-I and MHC-II restricted epitopes are provided in SEQ.ID.NO.3 and SEQ.ID.NO.4. In addition to the entire sequences provided in SEQ.ID.NO.3 and SEQ.ID.NO.4, the present invention also relates to fragments or variants of the amino acid sequences. Fragments of SEQ.ID.NO.3 and/or SEQ.ID.NO.4 include, but are not limited to

| | |
|---|---|
| LHWLMSVYVVELLRS, | (SEQ.ID.NO.17) |
| LFFYRKSVWSKLQSI, | (SEQ.ID.NO.18) |
| TSLRFIPKPDGLRP, | (SEQ.ID.NO.19) |
| RPGLLGASVLGLDDI, | (SEQ.ID.NO.20) |
| FAGIRRDGLLLRLVD, | (SEQ.ID.NO.21) |
| YGCVVNLRKTVVNFP, | (SEQ.ID.NO.22) |
| GTAFVQMPAHGLFPW, | (SEQ.ID.NO.23) |
| WCGLLLDTRTLEVQS, | (SEQ.ID.NO.24) |
| AKTFLRTLVRGVPEY, | (SEQ.ID.NO.25) |
| RPIVNMDYVVGARTFRREKR, | (SEQ.ID.NO.26) |
| LYFVKVDVTGAYDT, | (SEQ.ID.NO.27) |
| CHSLFLDLQVNSLQT, | (SEQ.ID.NO.28) |
| AKFLHWLMSVYVVEL, | (SEQ.ID.NO.29) |
| LMSVYVVELLRSFFY, | (SEQ.ID.NO.30) |
| MSVYVVELLRSFFYV, | (SEQ.ID.NO.31) |
| YVVELLRSFFYVTET, | (SEQ.ID.NO.32) |
| VELLRSFFYVTETTF, | (SEQ.ID.NO.33) |
| SFFYVTETTFQKNRL, | (SEQ.ID.NO.34) |
| KNRLFFYRKSVWSKL, | (SEQ.ID.NO.35) |
| KSVWSKLQSIGIRQH, | (SEQ.ID.NO.36) |
| WSKLQSIGIRQHLKR, | (SEQ.ID.NO.37) |
| QSIGIRQHLKRVQLR, | (SEQ.ID.NO.38) |
| SIGIRQHLKRVQLRE, | (SEQ.ID.NO.39) |
| RQHLKRVQLRELSEA, | (SEQ.ID.NO.40) |
| RPALLTSRLRFIPKP, | (SEQ.ID.NO.41) |
| PDGLRPIVNMDYVVG, | (SEQ.ID.NO.42) |
| LRPIVNMDYVVGART, | (SEQ.ID.NO.43) |
| RPIVNMDYVVGARTF, | (SEQ.ID.NO.44) |
| NMDYVVGARTFRREK, | (SEQ.ID.NO.45) |
| ARTFRREKRAERLTS, | (SEQ.ID.NO.46) |
| AERLTSRVKALFSVL, | (SEQ.ID.NO.47) |
| VKALFSVLNYERARR, | (SEQ.ID.NO.48) |
| LFSVLNYERARRPGL, | (SEQ.ID.NO.49) |
| ASVLGLDDIHRAWRT, | (SEQ.ID.NO.50) |
| HRAWRTFVLRVRAQD, | (SEQ.ID.NO.51) |
| WRTFVLRVRAQDPPP, | (SEQ.ID.NO.52) |
| VLRVRAQDPPPELYF, | (SEQ.ID.NO.53) |
| ELYFVKVDVTGAYDT, | (SEQ.ID.NO.54) |
| TYCVRRYAVVQKAAH, | (SEQ.ID.NO.55) |
| VRRYAVVQKAAHGHV, | (SEQ.ID.NO.56) |
| HGHVRKAFKSHVSTL, | (SEQ.ID.NO.57) |
| RKAFKSHVSTLTDLQ, | (SEQ.ID.NO.58) |
| LTDLQPYMRQFVAHL, | (SEQ.ID.NO.59) |
| QPYMRQFVAHLQETS, | (SEQ.ID.NO.60) |
| TSPLRDAVVIEQSSS, | (SEQ.ID.NO.61) |
| RDAVVIEQSSSLNEA, | (SEQ.ID.NO.62) |
| SGLFDVFLRFMCHHA, | (SEQ.ID.NO.63) |
| LFDVFLRFMCHHAVR, | (SEQ.ID.NO.64) |
| FDVFLRFMCHHAVRIRGK, | (SEQ.ID.NO.65) |
| HHAVRIRGKSYVQCQ, | (SEQ.ID.NO.66) |
| GKSYVQCQGIPQGSI, | (SEQ.ID.NO.67) |
| RDGLLLRLVDDFLLVTP, | (SEQ.ID.NO.68) |
| DFLLVTPHLTHAKTFLRTLV, | (SEQ.ID.NO.69) |
| KTFLRTLVRGVPEYG, | (SEQ.ID.NO.70) |
| AHGLFPWCGLLLDTRTLEVQ, | (SEQ.ID.NO.71) |
| TLEVQSDYSSYARTSIRAS, | (SEQ.ID.NO.72) |
| QSDYSSYARTSIRAS, | (SEQ.ID.NO.73) |
| RTSIRASLTFNRGFKAGRNM, | (SEQ.ID.NO.74) |
| RRKLFGVLRLKCHSLFLD, | (SEQ.ID.NO.75) |
| HSLFLDLQVNSLQTVCTNIY, | (SEQ.ID.NO.76) |
| RTSIRASLTFNRGFK, | (SEQ.ID.NO.77) |
| RRKLFGVLRLKCHSLFLDLQ, | (SEQ.ID.NO.80) |
| LMSVYVVEL, | (SEQ.ID.NO.82) |
| YMRQFVAHL, | (SEQ.ID.NO.83) |
| LLLRLVDDF, | (SEQ.ID.NO.84) |
| FLRTLVRGV, | (SEQ.ID.NO.85) |
| GLLLDTRTLEV, | (SEQ.ID.NO.86) |
| ASLTFNRGF, and | (SEQ.ID.NO.87) |
| FLDLQVNSL, | (SEQ.ID.NO.88) |
| LYFVKVDVTGAYDTI, | (SEQ.ID.NO.89, hTRT706) |
| LFDVFLRFMCHHAVRIRGK, | (SEQ.ID.NO.90, hTRT805) |
| FAGIRRDGLLLRLVD, | (SEQ.ID.NO.91, hTRT854) |
| WCGLLLDTRTLEVQS, | (SEQ.ID.NO.92, hTRT930) |
| RTSIRASLTFNRGFK, and | (SEQ.ID.NO.93, hTRT951) |
| RRKLFGVLRLKCHSLFLDL. | (SEQ.ID.NO.94, hTRT971) |

A. Variants of hTRT

Amino acid sequence variants of the hTRT polypeptides can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This can include the insertion of an immunoreactive epitope or simply a single residue. Terminal additions, called fusion proteins, are discussed below.

Substitutional variants typically contain the exchange of one amino acid for another at one or more sites within the protein, and are designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids are substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes are made in the DNA sequences of genes without appreciable loss of their biological utility or activity, as discussed below.

In making such changes, the hydropathic index of amino acids are considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids are substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those that are within ±1 are particularly preferred, and those within ±0.5 are even more particularly preferred.

It is also understood in the art that the substitutions of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0+1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take several of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles are used, in conjunction with the principles outline above, to engineer second generation molecules having many of the natural properties of hTRT proteins, but with altered and even improved characteristics.

B. Domain Switching

Domain switching involves the generation of chimeric molecules using different but, in this case, related polypeptides. By comparing various hTRT proteins, one can make predictions as to the functionally significant regions of these molecules. It is possible, then, to switch related domains of these molecules in an effort to determine the criticality of these regions to hTRT function. These molecules can have additional value in that these “chimeras” can be distinguished from natural molecules, while possibly providing the same function.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Proteins

It is desirable to purify hTRT polypeptides, proteins or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest is further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

There is no general requirement that the protein or peptide always be provided in their most purified state. Indeed, it is contemplated that less substantially purified products will have utility in certain embodiments. Partial purification is accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification can have advantages in total recovery of protein product, or in maintaining the activity of an expressed protein.

E. Synthetic Peptides

The polypeptides of the invention can also be synthesized in solution or on a solid support in accordance with conventional techniques. Various automatic synthesizers are commercially available and can be used in accordance with known protocols. See, for example, Stewart and Young (1984); Tam et al., (1983); Merrifield (1986); and Barany and Merrifield (1979), each incorporated herein by reference. Alternatively, recombinant DNA technology is employed wherein a nucleotide sequence which encodes a peptide of the invention is inserted into an expression vector, transformed or transfected into an appropriate host cell and cultivated under conditions suitable for expression.

F. Antigen Compositions

The present invention also provides for the use of hTRT proteins or polypeptides as antigens for the immunization of animals relating to the production of antibodies. It is envisioned that hTRT or portions thereof, will be coupled, bonded, bound, conjugated or chemically-linked to one or more agents via linkers, polylinkers or derivatized amino acids. This is performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

G. Antibody Production

In certain embodiments, the present invention provides antibodies that bind with high specificity to the hTRT polypeptides provided herein.

Monoclonal antibodies (MAbs) are recognized to have certain advantages, e.g., reproducibility and large-scale production, and their use are generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, humanized antibodies are also contemplated, as are chimeric antibodies from mouse, rat, or other species, bearing human constant and/or variable region domains, bispecific antibodies, recombinant and engineered antibodies and fragments thereof.

Polyclonal antibodies are prepared by immunizing an animal with an immunogenic hTRT composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition can vary in its immunogenicity. It is often necessary therefore to boost the host immune system, as is achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that are used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens can even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it is desirable to co-administer biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/M²) (Johnson/Mead, NJ), cytokines such as y-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies were monitored by sampling blood of the immunized animal at various points following immunization.

A second, booster injection, is also given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies (MAb) are be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified hTRT protein, polypeptide, peptide or domain, be it a wild-type or mutant composition. The immunizing composition is administered in a manner effective to stimulate antibody producing cells.

Following immunization, somatic cells with the potential for producing antibodies, specifically B lymphocytes (B cells), are selected for use in the MAb generating protocol. These cells are obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells is used, as are known to those of skill in the art (Goding, pp. 65-66, 1986; Campbell, 1984). For example, where the immunized animal is a mouse, one can use P3-X63/Ag8, X63-Ag8.653, NS1/1.Ag 41, Sp210-Ag14, FO, NSO/U, MPC-11, MPC11-X45-GTG 1.7 and S194/5XX0 Bul; for rats, one can use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that is used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion can vary from about 20:1 to about 1:1, respectively, in the presence of an agent or agents (chemical or electrical) that promote the fusion of cell membranes. Fusion methods using Sendai virus are described by Kohler and Milstein (1975; 1976), and those using polyethylene glycol (PEG), such as 37% (v/v) PEG, by Gefter et al., (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines are exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means are further purified, if desired, using filtration, centrifugation and various chromatographic methods such as HPLC or affinity chromatography. Fragments of the monoclonal antibodies of the invention can be obtained from the monoclonal antibodies so produced by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach can be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

II. Expression Constructs

Also, provided in the present invention is polynucleotide sequences encoding the hTRT polypeptides of the present invention. These polynucleotide sequences, SEQ.ID.NO.1 and SEQ.ID.NO.2, encode the polypeptide sequences of SEQ.ID.NO.3 and SEQ.ID.NO.4.

In certain embodiments, the present invention involves the manipulation of genetic material to produce expression constructs that encode hTRT epitopes. Such methods involve the generation of expression constructs containing, for example, a heterologous nucleic acid sequence encoding an hTRT epitope of interest and a means for its expression, replicating the vector in an appropriate helper cell, and obtaining the peptides produced therefrom.

One skilled in the art is cognizant that it is not necessary that the polynucleotide sequence encode a full-length protein. It is simply necessary that the expressed protein comprise an epitope, which elicits the desired immune response when processed in antigen presenting cells.

The polynucleotide sequences encoding at least one epitope of hTRT are selected epitopes that are contained in SEQ.ID.NO.1 and SEQ.ID.NO.2. These sequences contain MHC-I and MHC-II restricted epitopes. Thus, one of skill in the art realizes that fragments of single epitopes contained within SEQ.ID.NO.1 or SEQ.ID.NO.2 can be used, for example, but not limited the following epitopes SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100.

A. Regulatory Elements

In addition to the polynucleotide sequences that encode the hTRT epitopes of the present invention, one skilled in the art is cognizant that other regulatory factors are necessary for the integration of the DNA, propagation of the DNA, transcription and translation of the DNA. Thus, the following description illustrates the other regulatory elements that can be included in the expression vector of the present invention.

1. Promoters

The particular promoter employed to control the expression of a polynucleotide sequence of interest is not believed to be important, so long as it is capable of directing the expression of the polynucleotide in the targeted cell. Thus, where a human cell is targeted, it is preferable to position the polynucleotide sequence coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene product is toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the subject, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Similarly, tissue specific promoters are used to effect transcription in specific tissues or cells so as to reduce potential toxicity or undesirable effects to non-targeted tissues. For example, promoters such as the HER-2 promoter and PSA associated promoter sequences.

In certain indications, it is desirable to activate transcription at specific times after administration of the gene therapy vector. This is done with such promoters as those that are hormone or cytokine regulatable. Cytokine and inflammatory protein responsive promoters that can be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-i acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that any of the above promoters alone or in combination with another can be useful according to the present invention depending on the action desired. In addition, this list of promoters should not be construed to be exhaustive or limiting, those of skill in the art will know of other promoters that are used in conjunction with the promoters and methods disclosed herein.

2. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Any promoter/enhancer combination (as per the Eukaryotic Promoter Data Base EPDB) can be used to drive expression of the gene. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence is employed such as human or bovine growth hormone and SV40 polyadenylation signals. Also contemplated as an element of the expression cassette is a terminator. These elements can serve to enhance message levels and to minimize read through from the cassette into other sequences.

4. Integration Sequences

In instances wherein it is beneficial that the expression vector replicate in a cell, the vector integrates into the genome of the cell by way of integration sequences, i.e., retrovirus long terminal repeat sequences (LTRs), the adeno-associated virus ITR sequences, which are present in the vector, or alternatively, the vector itself comprises an origin of DNA replication and other sequence which facilitate replication of the vector in the cell while the vector maintains an episomal form. For example, the expression vector can optionally comprise an Epstein-Barr virus (EBV) origin of DNA replication and sequences which encode the EBV EBNA-1 protein in order that episomal replication of the vector is facilitated in a cell into which the vector is introduced. For example, DNA constructs having the EBV origin and the nuclear antigen EBNA-1 coding are capable of replication to high copy number in mammalian cells and are commercially available from, for example, Invitrogen (San Diego, Calif.).

It is important to note that in the present invention it is not necessary for the expression vector to be integrated into the genome of the cell for proper protein expression. Rather, the expression vector can also be present in a desired cell in the form of an episomal molecule. For example, there are certain cell types in which it is not necessary that the expression vector replicate in order to express the desired protein. These cells are those which do not normally replicate and yet are fully capable of gene expression. An expression vector is introduced into non-dividing cells and express the protein encoded thereby in the absence of replication of the expression vector.

5. Internal Ribosome Binding Sites

In certain embodiments of the invention, the use of internal ribosome entry sites (IRES) elements are used to create multigene, or polycistronic messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

B. Selectable and Screenable Markers

In certain embodiments of the invention, transformed cells of the present invention is identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) are utilized. One of skill in the art would also know how to employ immunologic markers, possibly in conjunction with FACS analysis. The marker used is not believed to be important, so long as it is capable of being expressed simultaneously with the polynucleotide encoding a gene product. Further examples of selectable and screenable markers are well known to one of skill in the art.

III. Methods of Gene Transfer

In order to mediate the effect of the transgene expression in a cell, it will be necessary to transfer the expression constructs of the present invention into a cell. Such transfer employs viral or non-viral methods of gene transfer. This section provides a discussion of methods and compositions of gene transfer.

A. Non-Viral Transfer

Several non-viral methods for the transfer of expression constructs into cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

1. Ex vivo Transformation

Methods for tranfecting vascular cells and tissues removed from an organism in an ex vivo setting are known to those of skill in the art. For example, cannine endothelial cells have been genetically altered by retroviral gene transfer in vitro and transplanted into a canine (Wilson et al., 1989). In another example, yucatan minipig endothelial cells were transfected by retrovirus in vitro and transplanted into an artery using a double-balloon catheter (Nabel et al., 1989). Thus, it is contemplated that cells or tissues are removed and transfected ex vivo using the polynucleotides of the present invention. In particular aspects, the transplanted cells or tissues are placed into an organism. Thus, it is well within the knowledge of one skilled in the art to isolate dendritic cells from a mammal, transfect the cells with the retrogen expression vector and then administer the transfected or transformed cells back to the mammal.

2. Injection

In certain embodiments, a polynucleotide is delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments of the present invention include the introduction of a polynucleotide by direct microinjection. Direct microinjection has been used to introduce polynucleotide constructs into Xenopus oocytes (Harland and Weintraub, 1985). The amount of the retrogen expression vector used varies upon the nature of the antigen as well as the organelle, cell, tissue or organism used

3. Electroporation

In certain embodiments of the present invention, a polynucleotide is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

4. Calcium Phosphate

In other embodiments of the present invention, a polynucleotide is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

5. DEAE-Dextran

In another embodiment, a polynucleotide is delivered into a cell using DEAE-dextran followed by polyethylene glycol. In this manner, reporter plasmids were introduced into mouse myeloma and erythroleukemia cells (Gopal, 1985).

6. Sonication Loading

Additional embodiments of the present invention include the introduction of a polynucleotide by direct sonic loading. LTK⁻ fibroblasts have been transfected with the thymidine kinase gene by sonication loading (Fechheimer et al., 1987).

7. Liposome-Mediated Transfection

Yet further, the expression construct is entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-potential lipid complexes are potential non-viral vectors for use in gene therapy.

Liposome-mediated nucleic acid delivery and expression of foreign DNA in vitro has been very successful. Using the β-lactamase gene, Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection. Also included are various commercial approaches involving “lipofection” technology.

In certain embodiments of the invention, the liposome is complexed with a hemagglutinating virus (HVJ). This has been shown to facilitate fusion with the cell membrane and promote cell entry of liposome-encapsulated DNA (Kaneda et al, 1989). In other embodiments, the liposome is complexed or employed in conjunction with nuclear nonhistone chromosomal proteins (HMG-1) (Kato et al., 1991). In yet further embodiments, the liposome is complexed or employed in conjunction with both HVJ and HMG-1. In that such expression constructs have been successfully employed in transfer and expression of nucleic acid in vitro and in vivo, then they are applicable for the present invention.

In other embodiments, the delivery vehicle comprises a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also is specifically delivered into a cell type such as prostate, epithelial or tumor cells, by any number of receptor-ligand systems with or without liposomes. For example, the human prostate-specific antigen (Watt et al., 1986) is used as the receptor for mediated delivery of a nucleic acid in prostate tissue.

8. Receptor Mediated Transfection

Still further, a polynucleotide is delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a polynucleotide-binding agent. Others comprise a cell receptor-specific ligand to which the polynucleotide to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a polynucleotide delivery vehicle component of a cell-specific polynucleotide targeting vehicle comprises a specific binding ligand in combination with a liposome. The polynucleotide(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a polynucleotide to cells that exhibit upregulation of the EGF receptor.

9. Microprojectile Bombardment

Microprojectile bombardment techniques can be used to introduce a polynucleotide into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles are coated with at least one polynucleotide and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles can contain DNA rather than be coated with DNA. DNA-coated particles can increase the level of DNA delivery via particle bombardment but are not, in and of themselves, necessary.

B. Viral Vector-Mediated Transfer

In certain embodiments, transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

2. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

3. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

4. Other Viral Vectors

Other viral vectors are employed as expression constructs in the present invention. Vectors derived from viruses such as vaccinia virus (Ridgeway, 1988; Baichwal and Sugden, 1986; Coupar et al., 1988) canary pox virus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

IV. Screening to Identify of MHC-II-Restricted Antigens

In specific embodiments of the present invention, it is provided a method to identify a polynucleotide sequence which encodes at least one MHC-II restricted epitope that is capable of activating CD4+ helper T cells. Specifically, the method comprises the steps of: introducing an expression vector into an antigen presenting cell to produce a transduced antigen presenting cell, wherein the expression vector comprises a polynucleotide promoter sequence, a polynucleotide encoding a signal sequence, a polynucleotide encoding a test polypeptide, a polynucleotide encoding a cell binding element and a polynucleotide encoding a dendritic cell receptor, all operatively linked; contacting the transduced antigen presenting cell with naïve T-cells or primed T-cells; and assessing whether any naïve T-cells or primed T cells are activated upon contact with the transduced antigen presenting cell wherein activation of said T-cells indicates that the polynucleotide encoding the test polypeptide is a gene or fragment thereof capable of activating CD4+ helper T cells. One skilled in the art is cognizant that the test polypeptide is a polypeptide that has yet to be identified as one that activates CD4+ T-cells.

First, a cDNA library is constructed using mRNA from selected cells, i.e., tumor cells. When cDNA is prepared from cells or tissue that express the polynucleotide sequences of interest at extremely high levels, the cDNA clones that contain the polynucleotide sequence can be selected with minimal effort. For less abundantly transcribed polynucleotide sequences, various methods can be used to enrich for particular mRNAs before making the library. Retroviruses are used as a vector for the library.

Specifically, retroviral libraries provide the ideal way to deliver a high-complexity library into virtually any mitotically active cell type for expression cloning. Because the viral particles infect with high efficiency, they deliver a more complex library than transfection-based methods. One skilled in the art realizes that any vector can be used for the library.

Once the cDNA library is constructed, the viral vectors are transfected into packaging cells. Next, immature DCs derived from monocytes are transduced with the recombinant vectors and efficiency is determined. Transduced DCs are co-cultured with expanded autologous CD4+ T-cells. Activation of the T-cells is an indication that the polypeptide is capable of activating CD4+ T-cells. The in vitro T-cell activation assay is adapted to be a high-throughput automated assay in order to facilitate the testing of many different test polynucleotide sequences at one time. One skilled in the art recognizes that the present invention can be manipulated to transduce cells with expression vectors containing a variety of possible epitope sequences. The transduced cells are placed in 96-well plates, containing naïve T-cells, and the activation of the T-cells is assessed by automated assessment of incorporation of radioactivity into the DNA of the T-cells, using technology readily available in clinical immunology. Positive clones are identified by ELISA (GM-CSF) or IL2 surface expression by flow cytometric array. The positive clone is PCR amplified and sequenced to determine the protein.

The human genome is screened to identify the polynucleotide sequences that encode proteins and epitopes that are recognized by CD4+ T-cells. These polynucleotide products are used for cancer therapy or to induce immune tolerance for autoimmune disease therapy, or gene therapy. This basic screening procedure provides for the identification of epitopes for designing small therapeutic molecules.

A skilled artisan is cognizant that this screening procedure can be modified to screen a variety of genomes, i.e., human, viral, bacterial, or parasitic. Construction of cDNA libraries are well known in the art. Thus, a skilled artisan is capable of utilizing this information to alter the present invention to identify antigens.

V. Methods of Eliciting an Immune Response.

Another embodiment of the present invention is a method to elicit an immune response directed against an antigen.

More particularly, this method utilizes the expression vector of the present invention to manipulate cells to produce endogenous antigens as if they were exogenous antigens. This novel antigen presentation strategy involves transducing cells with a novel recombinant expression vector to produce and secrete a fusion protein consisting of an antigen and a cell-binding element. The secreted fusion protein is endocytosed or “retrogradely” transported into antigen presenting cells via receptor-mediated endocytosis (Daeron, 1997; Serre et al., 1998; Ravetch et al., 1993). As a result, the fusion protein, or “retrogen” as termed in the present disclosure because of its retrograde transport following secretion, is processed in the endosomal pathway and is presented on the cell surface of the antigen presenting cells as an MHC-II restricted exogenous antigenic fragments even though it has been produced endogenously. The MHC-II bound antigenic fragments of the antigen on the surface of the antigen presenting cells activate CD4+-T-cells that in turn stimulate CD8+T-cells and macrophages, as well as B-cells to induce both cellular and humoral immunity.

It has also been discovered in the present invention that the retrogen protein is also processed in the cytosolic pathway during the fusion protein synthesis, secretion and endocytosis and become associated with MHC-I on the surface of the antigen presenting cells to directly activate CD8+ T-cells. Activation of CD8+ T cells by internalized antigens is described in the art and for example, in Kovacsovics-Bankowski et al., 1995. In addition, as noted above and described in more detail elsewhere herein, B cells are activated by the secreted retrogen. Thus, B cell activation is enhanced markedly in the present system in that CD4+ cells also activates B cells. Thus, this strategy uses a unifying mechanism to activate all of the arms of the immune system.

In specific embodiments, the expression vector is introduced into a cell to produce a transduced cell. Expression of the retrogen protein in the cells results in secretion of the retrogen protein from the cells. Secreted retrogen protein can then be taken up by antigen presenting cells in the mammal for processing therein and expression therefrom as a MHC-I or a MHC-II complex. Thus, one skilled in the art realizes that the transduced cell or first cell, secretes the antigen and the secreted antigen is internalized into a cell, a second cell, either the same cell or a different cell. When the eukaryotic cell is an antigen presenting cell, the retrogen protein is expressed therein, secreted therefrom and can reenter the cell for processing and antigenic MHC presentation. When the eukaryotic cell is not an antigen presenting cell, the cell expresses and secretes the retrogen protein, which is subsequently taken up by an antigen presenting cell for antigenic MHC presentation. Non-antigen presenting cells useful in the invention include any cell which does not process antigens for MHC presentation. Antigen presenting cells include dendritic cells (DC), macrophages, monocytes and the like. Tumor cells, which are also included, are cells, which are or are not capable of processing antigens for MHC presentation.

It is also contemplated that the polypeptides of the present invention can be pulsed into the antigen presenting cells, which can then be administered to a subject.

VI. Treatment of Hyperproliferative Diseases

The present invention contemplates the treatment of a hyperproliferative disease. It also contemplates the use of the present invention to modulate a hyperproliferative disease. It is envisioned that the present invention is directed at the use of the hTRT polynucleotide and/or polypeptide sequences to treat subjects with hyperproliferative diseases such that these subjects are conferred a therapeutic benefit as a result of the treatment. Thus, a therapeutic benefit refers to a result that promotes or enhances the well-being of the subject with respect to the medical treatment of his/her hyperproliferative disease. A list of non-exhaustive examples of this includes extension of the subject's life by any period of time; decrease or delay in the neoplastic development of the disease; decrease in hyperproliferation; reduction in tumor growth; delay of metastases; reduction in the proliferation rate of a cancer cell, tumor cell, or any other hyperproliferative cell; induction of apoptosis in any treated cell or in any cell affected by a treated cell; and a decrease in pain to the subject that can be attributed to the subject's condition.

Treatment regimens may vary as well, and often depend on tumor type, tumor location, disease progression, and health and age of the subject. Obviously, certain types of tumor will require more aggressive treatment, while at the same time, certain subjects cannot tolerate more taxing protocols. The clinician will be best suited to make such decisions based on the known efficacy and toxicity (if any) of the therapeutic formulations.

In some embodiments, a hyperproliferative disease is further defined as cancer. Examples of cancer contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, bladder cancer.

Yet further, the hyperproliferative disease, includes but is not limited to neoplasms. A neoplasm is an abnormal tissue growth, generally forming a distinct mass that grows by cellular proliferation more rapidly than normal tissue growth. Neoplasms show partial or total lack of structural organization and functional coordination with normal tissue. These can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoietic structures (structures pertaining to the formation of blood cells) including components of the immune system, are called leukemias, lymphomas and myelomas. A tumor is the neoplastic growth of the disease cancer. As used herein, a “neoplasm”, also referred to as a “tumor”, is intended to encompass hematopoietic neoplasms as well as solid neoplasms. Examples of neoplasms include, but are not limited to melanoma, non-small cell lung, small-cell lung, lung, hepatocarcinoma, retinoblastoma, astrocytoma, gliobastoma, gum, tongue, leukemia, neuroblastoma, head, neck, breast, pancreatic, prostate, renal, bone, testicular, ovarian, mesothelioma, sarcoma, cervical, gastrointestinal, lymphoma, brain, colon, bladder, myeloma, or other malignant or benign neoplasms.

Other hyperproliferative diseases include, but are not limited to neurofibromatosis, rheumatoid arthritis, Wegener's granulomatosis, Kawasaki's disease, lupus erathematosis, midline granuloma, inflammatory bowel disease, osteoarthritis, leiomyomas, adenomas, lipomas, hemangiomas, fibromas, vascular occlusion, restenosis, atherosclerosis, pre-neoplastic lesions in the mouth, prostate, breast, lung, etc., carcinoma in situ, oral hairy leukoplakia, or psoriasis, and pre-leukemias, anemia with excess blasts, and myelodysplastic syndrome.

In specific embodiments, the hyperproliferative disease is further defined as an immune-mediated disease. Immune-mediated diseases include, but are not limited to rheumatoid arthritis or inflammatory bowel disease.

A. Genetic Based Therapies

Specifically, the present invention intends to provide, to a cell, an expression construct capable of expressing a MHC-I and/or MHC-II restricted hTRT epitope. The lengthy discussion of expression vectors and the genetic elements employed herein are incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the subject. Similar figures are extrapolated for liposomal or other non-viral formulations by comparing relative uptake efficiencies. Formulation as a pharmaceutically acceptable composition is discussed below.

B. Protein Therapy

Another therapy approach is the provision, to a mammal, of a polypeptide of the present invention. The protein is produced by recombinant expression means. Formulations can be selected based on the route of administration and purpose including, but not limited to, liposomal formulations and classic pharmaceutical preparations. It is also envisioned that the present invention is used for peptide-based immunizations.

Yet further, it is envisioned that antibodies to the polypeptides can be administered to a subject. One of skill in the art is well aware that antibodies that bind with high specificity to the hTRT polypeptides provided herein can be produced. The techniques for preparing and using various antibody-based constructs and fragments are well known in the art. The discussion of antibodies employed herein is incorporated into this section by reference.

C. Cell Based Therapy

Another therapy that is contemplated is the administration of transduced antigen presenting cells. The antigen presenting cells are transduced in vitro or ex vivo. Formulation as a pharmaceutically acceptable composition is discussed below. One skilled in the art is cognizant that the antigen presenting cells can be transduced with peptides or polynucleotides encoding the peptides of the present invention.

Yet further, it is envisioned that the tumor cells can also be transduced with the peptides or polynucleotides encoding the peptides of the present invention.

In a further embodiment, the polypeptides of the present invention are pulsed into antigen presenting cells and/or tumor cells. The pulsed antigen-presenting cells and/or tumor cells are then administered to a subject.

D. Combination Treatments

In order to increase the effectiveness of the hTRT polypeptides, antibodies, nucleic acids, transgenes, or expression vectors, it is desirable to combine these compositions with other agents effective in the treatment of hyperproliferative disease, such as anti-cancer agents, or with surgery. An anti-cancer agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process involves contacting the cells with the expression construct and the agent(s) or multiple factor(s) at the same time. This is achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that hTRT gene therapy could be used similarly in conjunction with chemotherapeutic, radiotherapeutic, immunotherapeutic or other biological intervention, in addition to other pro-apoptotic or cell cycle regulating agents.

Alternatively, the gene therapy precedes or follows the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one contacts the cell with both modalities within about 12-24 h of each other and, more preferably, within about 6-12 h of each other. In some situations, it is desirable to extend the time period for treatment significantly, however, where several d (2, 3, 4, 5, 6 or 7) to several wk (1, 2, 3, 4, 5, 6, 7 or 8) lapse between the respective administrations.

Administration of the therapeutic expression constructs of the present invention to a subject will follow general protocols for the administration of chemotherapeutics, taking into account the toxicity, if any, of the vector. It is expected that the treatment cycles would be repeated as necessary. It also is contemplated that various standard therapies, as well as surgical intervention, are applied in combination with the described hyperproliferative cell therapy.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

Immunotherapeutics, generally, rely on the use of immune effector cells and molecules to target and destroy cancer cells. The immune effector is, for example, an antibody specific for some marker on the surface of a tumor cell. The antibody alone can serve as an effector of therapy or it can recruit other cells to actually effect cell killing. The antibody also can be conjugated to a drug or toxin (chemotherapeutic, radionuclide, ricin A chain, cholera toxin, pertussis toxin, etc.) and serve merely as a targeting agent. Alternatively, the effector can be a lymphocyte carrying a surface molecule that interacts, either directly or indirectly, with a tumor cell target. Various effector cells include cytotoxic T cells and NK cells.

a. Passive Immunotherapy

A number of different approaches for passive immunotherapy of cancer exist. They are broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the subject. However, their application is somewhat limited by their scarcity and have so far only been administered intralesionally. Human monoclonal antibodies to ganglioside antigens have been administered intralesionally to subjects suffering from cutaneous recurrent melanoma (Irie & Morton, 1986). Regression was observed in six out of ten subjects, following, daily or weekly, intralesional injections. In another study, moderate success was achieved from intralesional injections of two human monoclonal antibodies (Irie et al., 1989).

It can be favorable to administer more than one monoclonal antibody directed against two different antigens or even antibodies with multiple antigen specificity. Treatment protocols also can include administration of lymphokines or other immune enhancers as described by Bajorin et al., (1988). The development of human monoclonal antibodies is described in further detail elsewhere in the specification.

b. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those subjects who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

c. Adoptive Immunotherapy

In adoptive immunotherapy, the subject's circulating lymphocytes, or tumor infiltrated lymphocytes, are isolated in vitro, activated by lymphokines such as IL-2 or transduced with genes for tumor necrosis, and re-administered (Rosenberg et al., 1988; 1989). To achieve this, one would administer to an animal, or human subject, an immunologically effective amount of activated lymphocytes in combination with an adjuvant-incorporated antigenic peptide composition as described herein. The activated lymphocytes will most preferably be the subject's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

4. Genes

In yet another embodiment, the secondary treatment is a secondary gene therapy in which a second therapeutic polynucleotide is administered before, after, or at the same time a first therapeutic polynucleotide encoding at least one hTRT epitope. Delivery of a vector encoding a hTRT epitope in conjunction with a second vector encoding one of the following gene products will have a combined anti-hyperproliferative effect on target tissues. Alternatively, a single vector encoding both genes is used. A variety of proteins are encompassed within the invention, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme can be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus can regulate Rb phosphorylation (Serrano et al., 1993; Serrano et al., 1995). Since the p16INK4 protein is a CDK4 inhibitor (Serrano, 1993), deletion of this gene can increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that can be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclXL, BclW, BclS, Mcl-1, Al, Bfl-1) or counteract Bcl-2 function and promote cell death (e.g., Bax, Bak, Bik, Bim, Bid, Bad, Harakiri).

5. Surgery

Approximately 60% of persons with cancer will undergo surgery of some type, which includes preventative, diagnostic or staging, curative and palliative surgery. Curative surgery is a cancer treatment that is used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention is used in conjunction with removal of superficial cancers, precancers, or incidental amounts of normal tissue.

Upon excision of part of all of cancerous cells, tissue, or tumor, a cavity is formed in the body. Treatment is accomplished by perfusion, direct injection or local application of the area with an additional anti-cancer therapy. Such treatment is repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments can be of varying dosages as well.

6. Other Agents

It is contemplated that other agents are used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL can be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a subject's tissue is exposed to high temperatures (up to 106° F.). External or internal heating devices are involved in the application of local, regional, or whole-body hyperthermia. Local hyperthermia involves the application of heat to a small area, such as a tumor. Heat is generated externally with high-frequency waves targeting a tumor from a device outside the body. Internal heat involves a sterile probe, including thin, heated wires or hollow tubes filled with warm water, implanted microwave antennae, or radiofrequency electrodes.

A subject's organ or a limb is heated for regional therapy, which is accomplished using devices that produce high energy, such as magnets. Alternatively, some of the subject's blood is removed and heated before being perfused into an area that will be internally heated. Whole-body heating is also implemented in cases where cancer has spread throughout the body. Warm-water blankets, hot wax, inductive coils, and thermal chambers are used for this purpose.

Hormonal therapy is also used in conjunction with the present invention or in combination with any other cancer therapy previously described. The use of hormones is employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VII. Formulations and Routes for Administration to Subjects

Where clinical applications are contemplated, it will be necessary to prepare pharmaceutical positions—nucleic acids, expression vectors, proteins or cells—in a form appropriate for the intended application. Generally, this entails preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a subject. Aqueous compositions of the present invention comprise an effective amount of the vector to cells, dissolved or dispersed in a pharmaceutically acceptable carrier or aqueous medium. Such compositions also are referred to as inocula. The phrase pharmaceutically or pharmacologically acceptable refers to molecular entities and compositions that do not produce adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances are well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention includes classic pharmaceutical preparations. Administration of these compositions according to the present invention will be via any common route so long as the target tissue is available via that route. This includes oral, nasal, buccal, rectal, vaginal or topical. Alternatively, administration is by orthotopic, intradermal, subcutaneous, intramuscular, intraperitoneal or intravenous injection. Such compositions would normally be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For oral administration, the compositions of the present invention are incorporated with excipients and used in the form of non-ingestible mouthwashes and dentifrices. A mouthwash is prepared incorporating the active ingredient in the required amount in an appropriate solvent, such as a sodium borate solution (Dobell's Solution). Alternatively, the active ingredient is incorporated into an antiseptic wash containing sodium borate, glycerin and potassium bicarbonate. The active ingredient also is dispersed in dentifrices, including: gels, pastes, powders and slurries. The active ingredient is added in a therapeutically effective amount to a paste dentifrice that includes water, binders, abrasives, flavoring agents, foaming agents, and humectants.

The compositions of the present invention are formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media, which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Generation of a Retrogen

cDNA from the mRNA of prostate tumor cells (DU145) were generated by cDNA synthesis kits (CLONTECH). cDNA with sizes of 400 bp or higher were digested with Sau3A I (Bio-Lab) after fractionation with SizeSep™ 400 columns (Amersham Pharmacia Biotech) and inserted into the BamH I-cut pSBFc-DS. Ligated DNA was then transformed into E. coli, and library quality was confirmed by examining dozens of individual clones with different insert sizes and DNA sequences. Individual colonies were then cultured individually and pooled for the isolation of plasmid DNA. Recombinant retroviral retrogen vectors (pools) were generated by transfection of the plasmid DNA pools into packaging cells with GeneJammer (Stratagene). Two or three days later, the culture medium containing the retroviral vectors was harvested, filtered, and then used for DC transduction or stored at −80° C.

### Example 2

### Cell Lines and Blood Donors

Prostate cancer cell line (LNCaP-FGC), breast cancer cell lines (BT-474 and MDA-MB231), melanoma cell lines (SK-MEL37 and NA-6-MEL), human leukemia cell lines (HL-60 and Jurkat), and the hTRT-negative cell line GM847 were from ATCC. HLA typing of peripheral blood donors and tumor lines was performed by PCR-SSP DNA-based procedures in the HLA, Flow and Diagnostic Immunology Laboratory of the Methodist Hospital (Houston, Tex.). Peripheral bloods were obtained from adult healthy donors with their consent (donor B15; B24; B22; B14; B16; and B05).

### Example 3

### Transduction of Cells

Human CD34+ cells were isolated from the umbilical cord blood of healthy neonates by using a CD34 isolation kit (Miltenyi Biotec). Naïve CD4+/CD45RA+ T-cells were also isolated from the same cord blood. CD34+ cells were cultured and expanded in StemSpan™ SFEM (StemCell Technologies) medium in the presence of 80 ng/ml Flt-3 ligand, 100 ng/ml SCF, 10 ng/ml IL-3, 20 ng/ml IL-6, 30 ng/ml TPO, and 25 ng/ml low density lipoproteins (R&D Systems and BioSources International). For retroviral transduction, CD34+ cells were placed in 24-well plates precoated with recombinant human fibronectin fragment (PanVera) at 2.5-5×10⁵ cells/well in the retroviral supernatant supplemented with 80 ng/ml Flt-3 ligand, 100 ng/ml SCF, 10 ng/ml IL-3, and 20 ng/ml IL-6 overnight. After 2-3 consecutive transductions, the cells were transferred into new 24-well plates and cultured in SFEM supplemented with GM-CSF (1000 U/ml, Immunex) and IL-4 (5 ηg/ml, BioSource International) for 6-8 days for (dendritic cell) DC differentiation. Mature DCs were then generated by adding TNF-α (25 ng/ml, R&D Systems) for 24-48 hr. DC Phenotypes were examined by staining with PE-conjugated anti-HLA-DR, -CD40, -CD54, -CD80, -CD83 and -CD86 mAbs (PharMingen).

### Example 4

### PBMC-Derived DC Culture

Human dendritic cells (DC) were prepared (Schroers et al., 2000). Briefly, PBMCs were isolated by Ficoll-Hypaque gradient centrifugation (Pharmacia), washed in phosphate-buffered saline (PBS), and resuspended in serum-free DC medium (CellGenix). After adherence to plastic for 2 hours, the adherent cell fraction was cultured in serum-free DC medium with 1,000 IU/ml recombinant human GM-CSF (rhGM-CSF; R&D Systems) and 1,000 IU/ml rhIL-4 (R&D Systems). On day 5, DC were matured by stimulating with a cytokine cocktail consisting of recombinant human tumor necrosis factor alpha (rhTNF-α; 10 ng/ml, R&D Systems), rhIL-1β (1,000 ng/ml, R&D Systems), rhIL-6 (10 ng/ml, R&D Systems), and prostaglandin E2 (PGE2; 1 μg/ml, Sigma). (Jonuleit et al., 1997).

### Example 5

### Antigen Pulsing of DC

Tumor cells (5×10⁷) were washed twice with PBS, resuspended in 2 ml of DC culture medium, and then lysed by 5 freeze-thaw cycles. The cells were sonicated for 10 minutes and then centrifuged at 15,000 g for 30 min (4° C.). Supernatant was recovered, aliquoted and stored at −70° C. until further use. 20 ul of the supernatant were added to a total of 5×10⁵ DC in 500 ul of DC culture medium. Proteins or peptides were then added to the DC culture at different concentrations. After overnight incubation, the pulsed DC were carefully washed with PBS and irradiated with 40 Gy prior to coculture with T cells.

### Example 6

### In vitro Priming of Naïve T Cells

The MF-DS retroviral vector coexpressed the MAGE-3 (a known class II-restricted tumor antigen)-Fc retrogen (MF) (Chaux et al., 1999; You et al., 2001) and DC-SIGN, while the HF-DS retroviral vector coexpressed a Hepatitis B virus e retrogen (HBe-Fc) (You et al., 2000) and DC-SIGN. Cord blood was used as a source of autologous CD34+-derived DC and CD4+ T-cells.

To prime naïve CD4+ T-cells, cord blood CD34+ cells were transduced by the retroviral vector MF-DS or HF-DS and differentiated into DC. Mature DC (1×10³/well) were then cocultured with autologous naïve CD4+/CD45RA+ T cells (1×10⁵/well). After two weeks of co-culture with re-stimulation, CD4+ T cell responses were assessed by analysis of [³H]-thymidine incorporation and IFN-γ secretion, after re-stimulation with autologous DC transduced with MF-DS. CD4+ T cells primed by MF-DS-transduced DC were stimulated with autologous DC transduced with MF-DS, HF-DS or untransduced DC. CD4+ T cells primed by MF-DS-transduced DC were stimulated with autologous DC pulsed with recombinant MAGE-3 and irrelevant HBe/cAg proteins (10 μg/ml) (You et al., 2001).

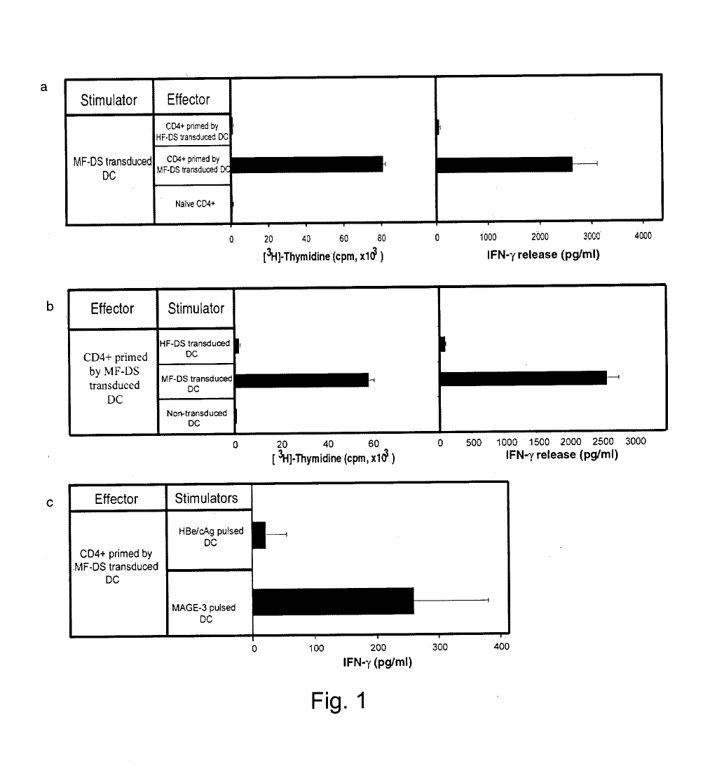

The data showed that CD4+ T-cells primed by the MF-DS-transduced DC actively proliferated and produced high levels of IFN-γ when stimulated with MF-DS-transduced DC (FIG. 1). These responses were specific, since MF-DS-transduced DC did not stimulate CD4+ T-cells primed by DC transduced with the irrelevant HBe-Fc retrogen (You et al., 2000) or naïve T-cells, and the CD4+ T-cells primed by MF-DS-transduced DC did not respond to DC transduced with the irrelevant HBe-Fc retrogen (FIG. 1). Moreover, the CD4+ T-cells primed by MF-DS-transduced DC responded to recombinant MAGE-3-pulsed autologous DC, but not to DC pulsed with the recombinant proteins HBe/cAg (FIG. 1). These results indicate that transduced DC coexpressing a retrogen and DC-SIGN can efficiently prime antigen-specific naïve human CD4+ T-cells in vitro.

### Example 7

### DC-Based Immunogenic Screening

DC-based immunogenetic screening approach was used to identify unknown class II-restricted TAA from a tumor retrogen library.

After several rounds of screening of 12,000 individual clones in the library, two sub-pools (5-VI-C and -D) containing genes capable of stimulating CD4+ T-cells were identified. The 40 individual clones in the sub-pools 5-VI-C and -D were individually transduced into autologous CD34+-derived DC. The responses of T-cells (2×10⁵) primed by the pool 5-transduced DC were examined with a [³H]-thymidine incorporation assay after stimulation with autologous DC (2×10⁴) transduced with individual clones.

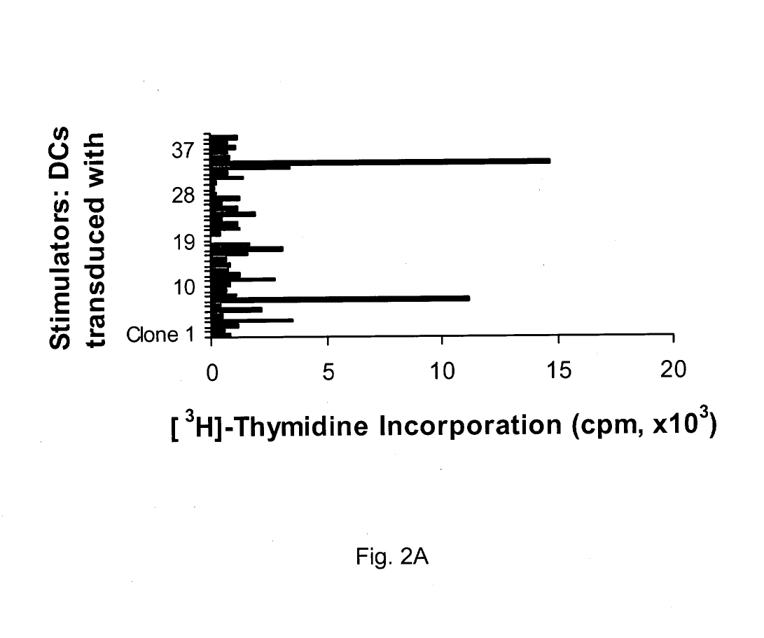

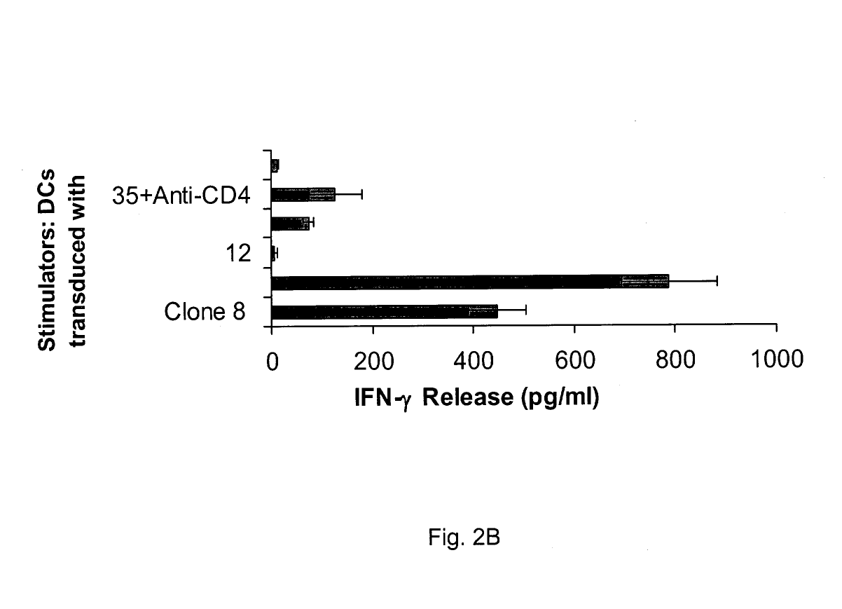

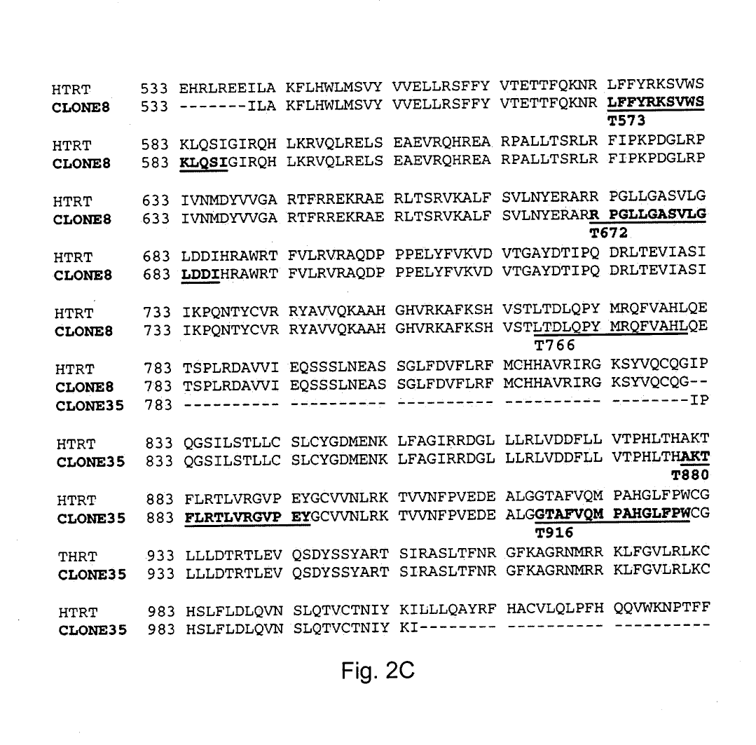

FIG. 2A shows that clones 8 and 35 induce strong T-cell response.

### Example 8

### Antibody Blocking

The T cells primed by the pool 5-transduced DC (2×10⁵) were co-cultured with autologous DC (2×10⁴) transduced with clone 8, 35, negative clone 12, or irrelevant HBeAg (You et al., 2000) in the presence or absence of anti-CD4 antibodies (30 mg/ml). Two days later, IFN-γ release in the T-cell cultures was examined by ELISA (R&D Systems).

Thus, anti-CD4 antibody blocking experiments demonstrated that the T-cell responses were mediated by CD4+ T-cells (FIG. 2B).

### Example 9

### DNA Sequencing and hTRT Identification

DNA sequencing of clones 8 (SEQ.ID.NO.1) and 35 (SEQ.ID.NO.2) revealed an open reading frame (ORF) in clone 8 that encodes a 291 amino acid (aa) residue polypeptide (SEQ.ID.NO.3) and an ORF in clone 35 that encodes a 171 aa polypeptide (SEQ.ID.NO.4) (FIG. 2C). By BLAST searching, the two clones were found to be 100% homologous with portions of the human telomerase reverse transcriptase (hTRT) (Nakamura et al., 1997) (FIG. 2C). Neither of the two clone sequences harbored a mutation. Thus, by screening the tumor retrogen library, a class II-restricted TAA candidate was identified, hTRT.

By analysis with TEPITOPE, a T cell epitope prediction program (Manici et al., 1999), Table I shows class-II-restricted epitopes in the clone 8 and 35 sequences of hTRT that were predicted to bind HLA DR3, DR4, and DR7 at the 3% prediction threshold.

TABLE 1
hTRT Peptides

| | |
|---|---|
| LHWLMSVYVVELLRS | (SEQ.ID.NO.17, T545) |
| LFFYRKSVWSKLQSI | (SEQ.ID.NO.18, T573) |
| TSRLRFIPKPDGLRP | (SEQ.ID.NO.19, T618) |
| RPGLLGASVLGLDDI | (SEQ.ID.NO.20, T672) |
| FAGIRRDGLLLRLVD | (SEQ.ID.NO.21, T844) |
| YGCVVNLRKTVVNFP | (SEQ.ID.NO.22, T894) |
| GTAFVQMPAHGLFPW | (SEQ.ID.NO.23, T916) |
| WCGLLLDTRTLEVQS | (SEQ.ID.NO.24, T930) |
| AKTFLRTLVRGVPEY | (SEQ.ID.NO.25, T880) |
| RPIVNMDYVVGARTFRREKR | (SEQ.ID.NO.26, T631) |
| LYFVKVDVTGAYDT | (SEQ.ID.NO.27, T706) |
| CHSLFLDLQVNSLQT | (SEQ.ID.NO.28, T983) |
| AKFLHWLMSVYVVEL | (SEQ.ID.NO.29) |
| LMSVYVVELLRSFFY | (SEQ.ID.NO.30) |
| MSVYVVELLRSFFYV | (SEQ.ID.NO.31) |
| YVVELLRSFFYVTET | (SEQ.ID.NO.32) |
| VELLRSFFYVTETTF | (SEQ.ID.NO.33) |
| SFFYVTETTFQKNRL | (SEQ.ID.NO.34) |
| KNRLFFYRKSVWSKL | (SEQ.ID.NO.35) |
| KSVWSKLQSIGIRQH | (SEQ.ID.NO.36) |
| WSKLQSIGIRQHLKR | (SEQ.ID.NO.37) |
| QSIGRQHLKRVQLR | (SEQ.ID.NO.38) |
| SIGIRQHLKRVQLRE | (SEQ.ID.NO.39) |
| RQHLKRVQLRELSEA | (SEQ.ID.NO.40) |
| RPALLTSRLRFIPKP | (SEQ.ID.NO.41) |
| PDGLRPIVNMDYVVG | (SEQ.ID.NO.42) |
| LRPIVNMDYVVGART | (SEQ.ID.NO.43) |
| RPIVNMDYVVGARTF | (SEQ.ID.NO.44) |
| NMDYVVGARTFRREK | (SEQ.ID.NO.45) |
| ARTFRREKRAERLTS | (SEQ.ID.NO.46) |
| AERLTSRVKALFSVL | (SEQ.ID.NO.47) |
| VKALFSVLNYERARR | (SEQ.ID.NO.48) |
| LFSVLNYERARRPGL | (SEQ.ID.NO.49) |
| ASVLGLDDIHRAWRT | (SEQ.ID.NO.50) |
| HRAWRTFVLRVRAQD | (SEQ.ID.NO.51) |
| WRTFVLRVRAQDPPP | (SEQ.ID.NO.52) |
| VLRVRAQDPPPELYF | (SEQ.ID.NO.53) |
| ELYFVKVDVTGAYDT | (SEQ.ID.NO.54) |
| TYCVRRYAVVQKAAH | (SEQ.ID.NO.55) |
| VRRYAVVQKAAHGHV | (SEQ.ID.NO.56) |
| HGHVRKAFKSHVSTL | (SEQ.ID.NO.57) |
| RKAFKSHVSTLTDLQ | (SEQ.ID.NO.58) |
| LTDLQPYMRQFVAHL | (SEQ.ID.NO.59) |
| QPYMRQFVAHLQETS | (SEQ.ID.NO.60) |
| TSPLRDAVVIEQSSS | (SEQ.ID.NO.61) |
| RDAVVIEQSSSLNEA | (SEQ.ID.NO.62) |
| SGLFDVFLRFMCHHA | (SEQ.ID.NO.63) |
| LFDVFLRFMCHHAVR | (SEQ.ID.NO.64) |
| FDVFLRFMCHHAVRIRGK | (SEQ.ID.NO.65) |
| HHAVRIRGKSYVQCQ | (SEQ.ID.NO.66) |
| GKSYVQCQGIPQGSI | (SEQ.ID.NO.67) |
| RDGLLLRLVDDFLLVTP | (SEQ.ID.NO.68) |
| DFLLVTPHLTHAKTFLRTLV | (SEQ.ID.NO.69) |
| KTFLRTLVRGVPEYG | (SEQ.ID.NO.70) |
| AHGLFPWCGLLLDTRTLEVQ | (SEQ.ID.NO.71) |
| TLEVQSDYSSYARTSIRAS | (SEQ.ID.NO.72) |
| QSDYSSYARTSIRAS | (SEQ.ID.NO.73) |
| RTSIRASLTFNRGFKAGRNM | (SEQ.ID.NO.74) |
| RRKLFGVLRLKCHSLFLD | (SEQ.ID.NO.75) |
| HSLFLDLQVNSLQTVCTNIY. | (SEQ.ID.NO.76) |
| RTSIRASLTFNRGFK | (SEQ.ID.NO.77, T951) |

The purity of the peptides was >90% by HPLC. Synthetic peptides were reconstituted in distilled water or DMSO at a concentration of 5 μg/ml.

### Example 10

### Peptide T-Cell Proliferation Assay and T-Cell Clone Establishment

Donor's peripheral blood mononuclear cells (PBMCs) were plated in 96-well plates (Costar) at 200,000 cells/well in AIM-V media (Gibco). Peptides were added into each well at the concentration of 20 μg/ml. After a week of incubation, the culture medium was removed and cells were resuspended in AIM-V media, seeded onto test plates and re-stimulated with autologous irradiated (2,000-6,000 rads) PBMC pulsed with the same peptides used in the primary stimulation (20 μg/ml). On day 2 of the re-stimulation, [³H]-thymidine (1 uCi/well) was added to the test plates, and its incorporation by T-cells was measured on day 3. Wells were scored as positive if the mean cpm for peptide-pulsed PBMC exceeded cpm for PBMC not exposed to peptides by at least 2.5 times. T-cell clones were established from positive T-cell lines by limiting dilution.

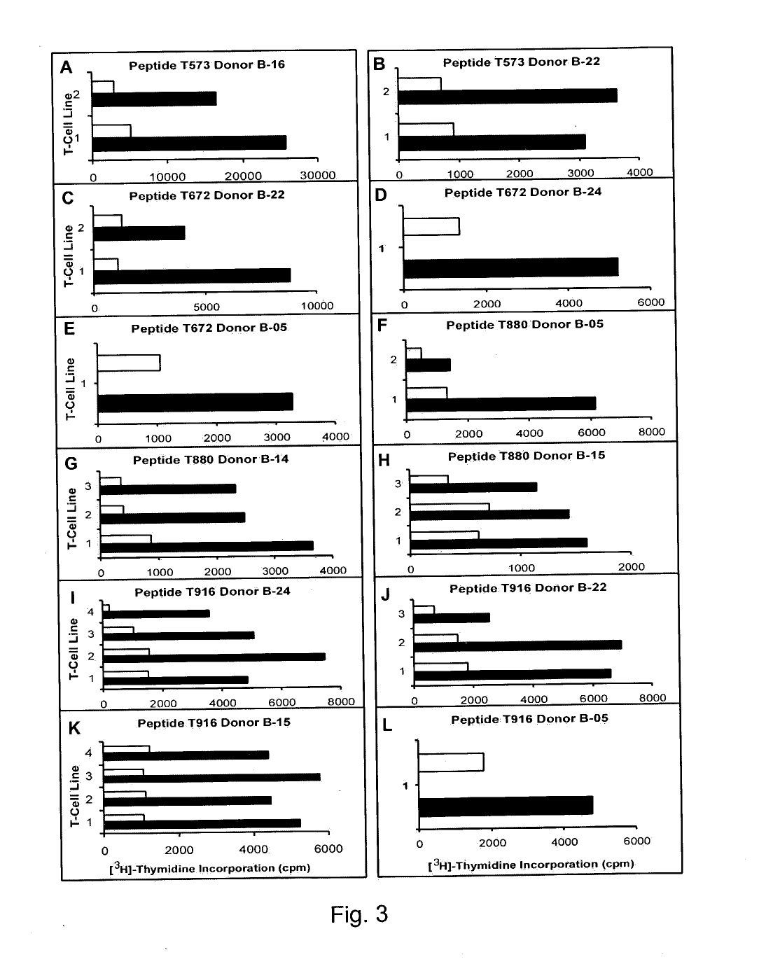

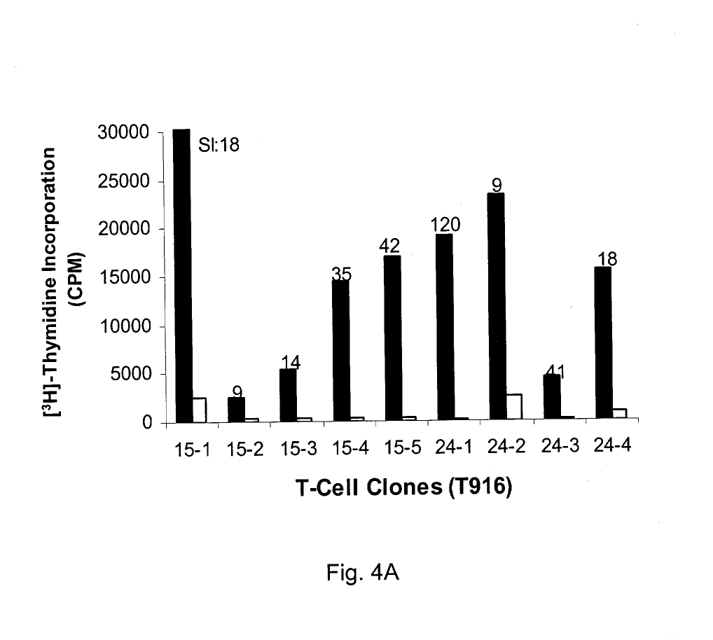

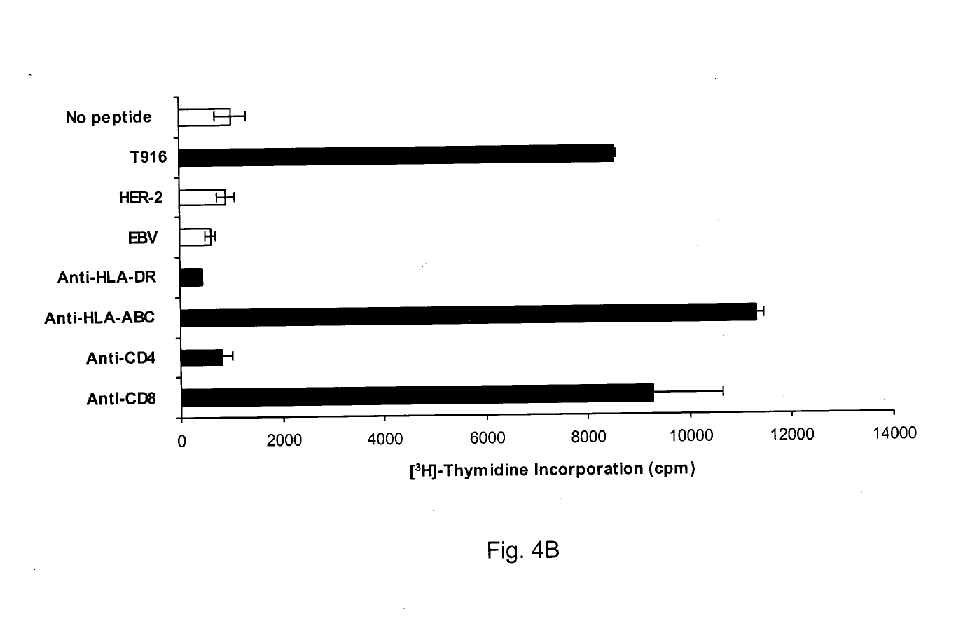

As summarized in FIG. 3A-FIG. 3L, four peptides (T573, T672, T880, and T916) elicited proliferative T-cell responses from the donors' T-cells, and therefore are class II-restricted epitopes.

[³H]-thymidine incorporation of different individual T-cell clones was measured after re-stimulation with autologous PBMC in the presence or absence of T916 (20 μg/ml) (FIG. 4B). Most T-cell clones strongly responded to autologous T916-pulsed PBMC with stimulation indexes ranging from 9 to 120 (FIG. 4B).

Two individual T-cell clones were generated from hTRT672-reactive T-cell lines from donors.

### Example 11

### Specificity of T-Cell Responses

The T916-positive T-cell clone, T631-positive T-cell or T672-positive T-cell (2×10⁴ cells/well) were re-stimulated with autologous PBMC-derived DC (1×10³/well) (Zhou et al., 1996) pulsed with T916, T631, T672 or irrelevant 15-mer peptides derived from HER-2 (SEQ.ID.NO.78, LSTDVGSCTLVCPLH) and EBV (SEQ.ID.NO.79, AYFMVFLQTHIFAEV) at the same concentration of 20 μg/ml in the presence of anti-HLA-DR (G46.4), anti-HLA-ABC (class I, G46.2.6), anti-CD4, or anti-CD8 (20 μg/ml, BD PharMingen). 48-72 hr later, GM-CSF release and [³H]-thymidine incorporation of the T-cells were measured.

The T-cell responses to T916 were inhibited by anti-HLA-DR and anti-CD4 antibodies, but not by anti-HLA-ABC (class I) and anti-CD8 antibodies, indicating that the observed responses were both CD4- and HLA-DR-restricted (FIG. 4C). The T-cell response was specific, because the T-cells did not respond to stimulation with irrelevant 15-mer peptides derived from HER-2 or Esptein-Barr virus (EBV) (FIG. 4C). Moreover, the T-cells responded to autologous PBMC pulsed with T916 in a dosage-dependent manner.

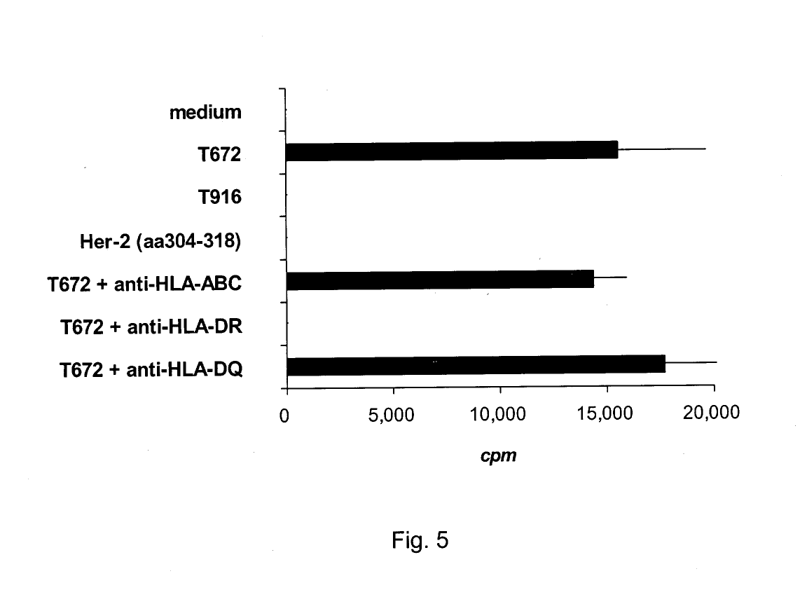

The responses of hTRT672 T-cells to the hTRT672 peptide were inhibited by an anti-HLA-DR antibody, but not by anti-HLA-ABC and anti-HLA-DQ antibodies, indicating that the observed response was HLA-DR-restricted. The T-cell response was specific, since the T-cells did not respond to stimulation with irrelevant 15-mer peptides derived from HER-2 or with the peptide hTRT916 (FIG. 5).

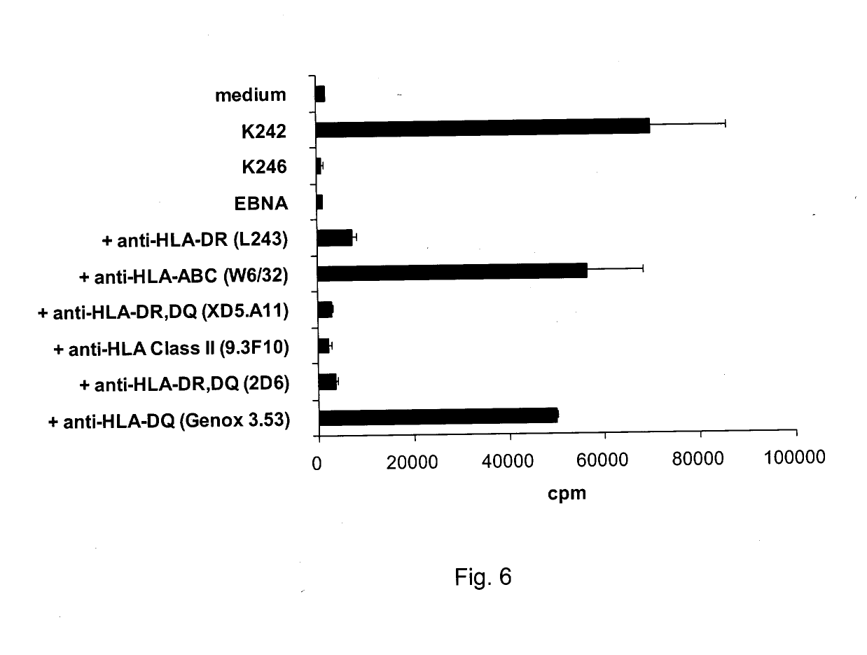

The primed T-cells did not responded to irrelevant 15 mer peptides from the EBV nuclear antigen 1 or from different hTRT sequence (hTRT573) (FIG. 6), indicating that the T-cell responses were specific. The responses of the T-cell clone to the HTRT631 peptide were also inhibited by anti-HLA-DR, anti-HLA class II antibodies, but not by anti-HLA-ABC (class I) and anti-HLA-DQ antibodies, indicating that the observed T-cell response was HLA-DR-restricted.

Thus, the above data illustrate that the T916, T631 and T672 are MHC-II restricted epitopes.

### Example 12

### Peptide Titration

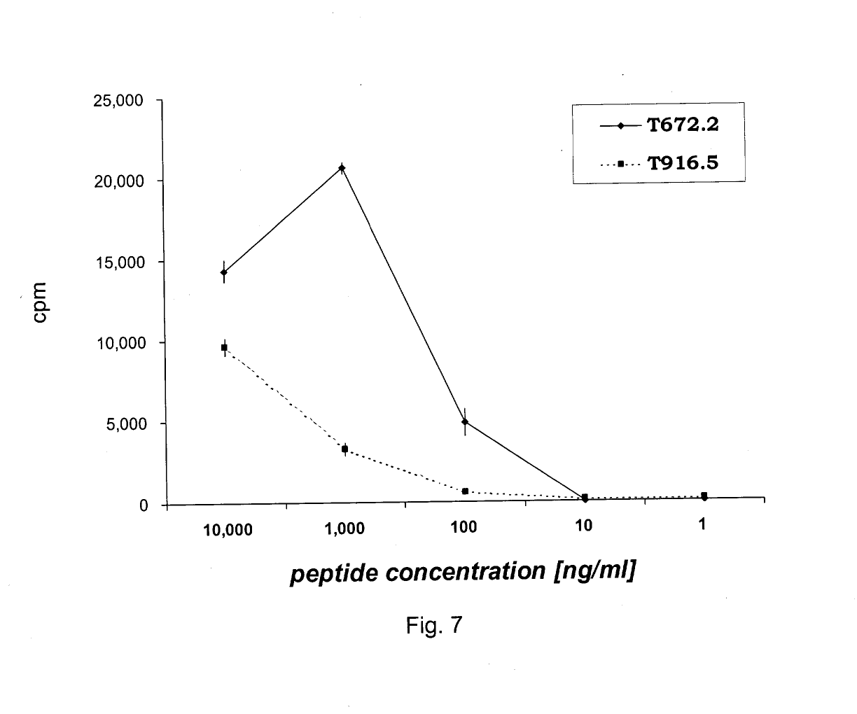

To evaluate the avidity of hTRT916 and hTRT672 T-cell clones for their ligands, peptide titration curves were generated with autologous PBMCs. The maximal cell proliferation of the hTRT916 T-cell clone was obtained at a peptide concentration of 10 μg/ml, compared with only at a peptide concentration of 1 μg/ml for the hTRT672 T-cell clone (FIG. 7). Thus, it is evident that the TCR of the hTRT672 T-cell clone exhibited higher avidity than the hTRT916 T-cell clone.

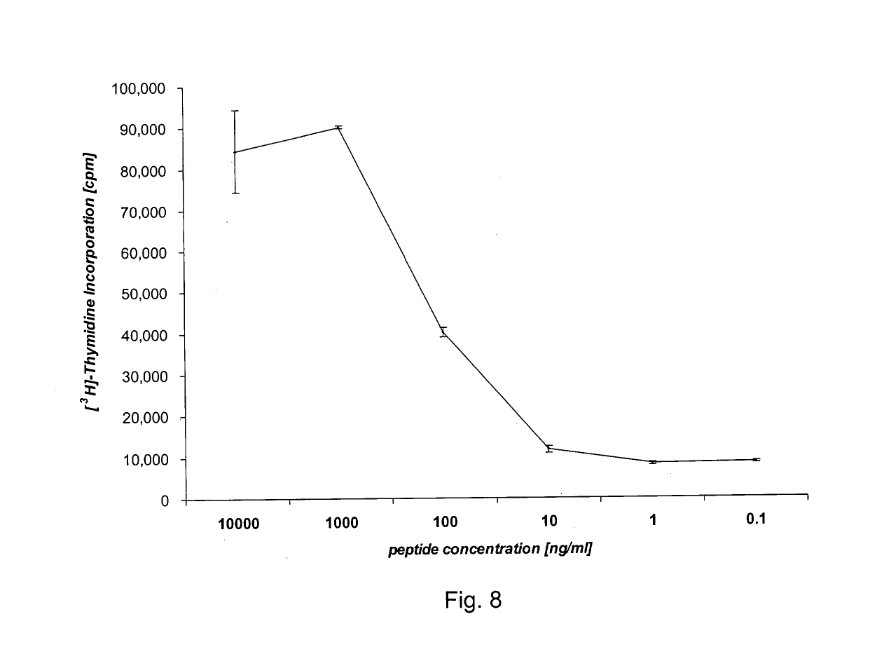

Yet further, the avidity of the hTRT631 T-cell clone for its ligand was also evaluated. Peptide titration curves were generated with autologous PBMCs. The half maximal cell proliferation of the hTRT631 T-cell clone was obtained at a peptide concentration of 0.1 μg/ml (FIG. 8).

### Example 13

### Flow Cytometric Analysis

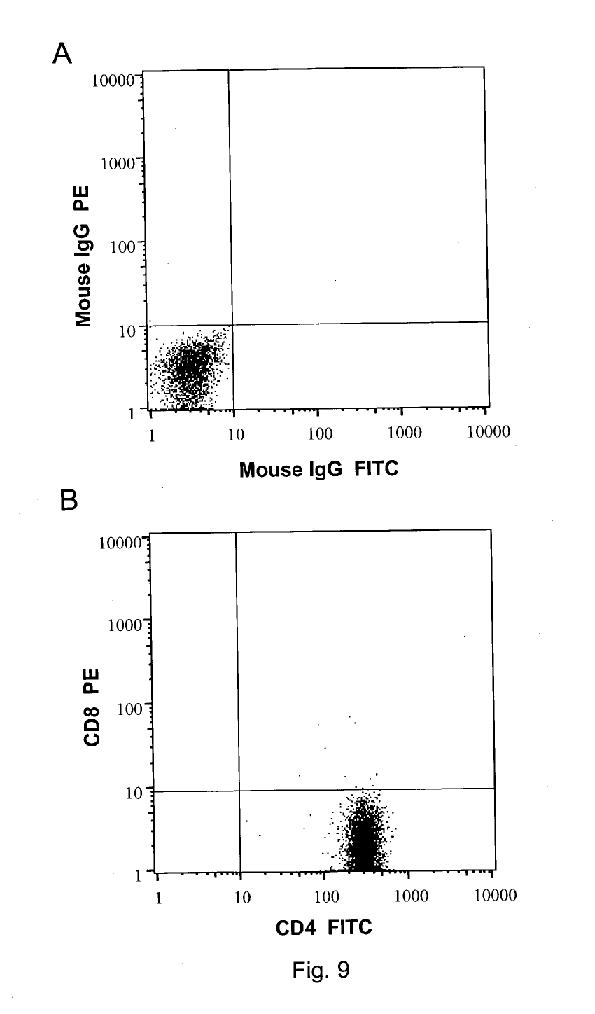

The T-cell clone was double-stained with anti-human CD4-FITC and CD8-PE antibodies or isotype controls (mouse IgG-FITC and IgG-PE) (BD PharMingen). The cells were then examined by flow cytometric analysis (Chen et al., 1997). More than 99% of the T-cell population were CD4-positive. Flow cytometric analysis verified that the T-cell clones were exclusively CD4-positive (FIG. 9A and FIG. 9B). Similar results were obtained from four different T916-specific T-cell clones. Taken together, the findings indicate that HLA-DR7-restricted epitopes reside in clones 8 and 35 and induce hTRT-specific human CD4+ T-cell responses.

### Example 14

### T-Cell Recognition of Natively Processed hTRT

A recombinant hTRT protein and an irrelevant Neu protein were produced and used to pulse PBMC-derived DC. Recombinant hTRT and Neu(extracellular domain)-Fc fusion proteins, were produced in SF9 insect cells by use of a baculovirus expression system (Gibco), purified by affinity binding to Protein A (Sigma), and tested by Western blot analysis with anti-hTRT (Santa Cruz) or anti-Neu (Oncogene) antibodies, respectively.

The T916-specific T-cells, T631-specific T-cells or T672-specific T-cells (2×10⁴/well) were stimulated with irradiated autologous PBMC-derived DC (Zhou et al., 1996) (2×10³/well) pulsed with recombinant hTRT-Fc proteins (1 μg/ml) in the presence or absence of anti-HLA-DR antibodies (20 μg/ml). The T-cell clones were also stimulated with DC pulsed with irrelevant recombinant Neu-Fc proteins (1 μg/ml). Recombinant hTRT Fc fusion proteins and Neu (extracellular domain)-Fc proteins were produced from transfected mammalian cells, purified with a Protein-A purification kit (Pierce), and confirmed by Western blot analysis with anti-hTRT (Santa Cruz) or anti-Neu (Oncogene) antibodies. 48 hr later, [³H]-thymidine incorporation of the T-cells was then measured.

The T916-specific T-cells were found to recognize the hTRT proteins after processing and presentation by autologous DC, as demonstrated by active T-cell proliferation. By contrast, the T-cells did not respond to the irrelevant protein Neu presented by autologous DC.

The hTRT631 T-cell clone recognized the hTRT protein after processing and presentation by autologous DC, as demonstrated by active T-cell proliferation and the secretion of GM-CSF. By contrast, the T-cell clone did not respond to the irrelevant Neu-Fc proteins presented by autologous DC. The T-cell response was inhibited by anti-HLA-DR antibodies.

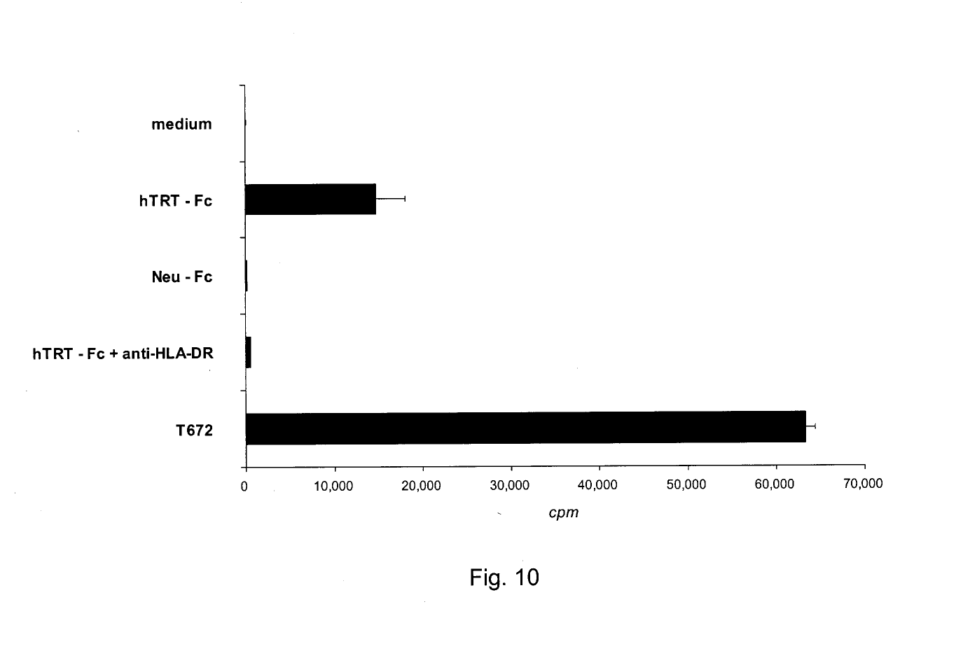

As shown in FIG. 10, the hTRT672 T cell clone recognized the hTRT protein after processing and presentation by autologous DC.

These results indicate that the synthetic peptide sequences, T916, T672 and T631, are recognized by the CD4+ T-cells and are processed.

### Example 15

### T-Cell Direct Responses to hTRT-Positive Cells

The T-cells (5×10³/well) were co-cultured with the irradiated HLA-DR7+/hTRT+ LCL cells (1×10⁴/well) in the presence or absence of the anti-HLA-DR antibody (20 μg/ml). IFN-γ production of the T-cells was monitored by ELISPOT assay and cell proliferation was measured by [³H]-thymidine incorporation compared with anti-DR sample.

The T916-specific CD4+ T-cells responded to the hTRT+/DR7+ LCL, as demonstrated by active proliferation and production of INF-γ. The T-cell response was drastically inhibited by the anti-HLA-DR antibody. In addition, the CD4+ T-cells did not respond to different class II genotypic hTRT+ LCL (DR3+). Thus, these results indicate that the CD4+ T-cells directly recognize hTRT peptide/MHC class II complexes on the surface of transformed cells.

### Example 16

### Frequency of hTRT-Specific T-Cells

The frequency of hTRT-specific CD4+ T-cells in humans was assessed. It is suggested that CD4+T-cells that recognize hTRT (self antigen) are largely clonally deleted during T-cell thymic selection.

T-cell precursor frequencies in HLA-DR7+ donors were calculated as the numbers of positive wells divided by the total numbers of T-cells in all wells tested. The T-cell precursor frequencies were 0.2-0.6×10⁻⁶ for T573, 0.2-0.6×10⁻⁶ for T672, 0.3-0.5×10⁻⁶ for T880, and 0.1-0.5×10⁻⁶ for T916. The hTRT precursor frequencies are comparable to published frequencies for other self-antigens (Zhang et al., 1994). Taken together, the data suggest that hTRT-specific CD4+ T-cell responses are readily induced and that precursors of hTRT-specific CD4+ T-cells are part of the normal human T-cell repertoire.

### Example 17

### CD4+ T-Cell Response Against Various Tumors

CD4+ T-cells react with antigen presenting cells (APCs) that take up and process the tumor antigen protein from apoptotic and dead tumor cells. Thus, the capacity of the hTRT672-reactive CD4+ T-cells clones to become activated when cocultured with APCs pulsed with lysates from different tumor types was tested.

The hTRT672 T-cell clones (2.5×10⁴/well) were stimulated with autologous DC (2.5×10³/well) pulsed with hTRT-positive tumor lysates, including the prostate tumor line LNCaP-FGC, breast tumor lines (BT-474 and MDA-MB231), melanoma lines (SK-MEL37 and NA-6-MEL), and leukemia lines (HL-60 and Jurkat), or hTRT-negative cell lysate (GM847). GM-CSF release and [³H]-thymidine incorporation by the T-cells was measured.

As shown in FIGS. 11A and 11B, the T-cells proliferated and secreted GM-CSF after stimulation with each of hTRT+ tumors of different tissues and organs, including prostate cancer (LNCaP-FGC), breast cancer (BT-474 and MDA-MB231), melanoma cell lines (SK-MEL37 and NA-6-MEL), but not to the stimulation with the hTRT-negative cells (GM847). This result indicates that the hTRT672 T-cells broadly recognize the hTRT epitope derived from tumors of different tissues and organs.

### Example 18

### Generation of Adenoviral Vectors

The replication defective (E1 and E3 deletions) adenoviral (Ad5) vector (Quantum Biotechnology, Toronto, Canada) was used to generate retrogen adenoviral vectors. The entire expression cassette (CMV promoter-shTRT-Fc-PolyA) derived from the LNC-shTRT-Fc vector1 was inserted into a transfer vector, and the resultant recombinant adenovirus ad-shTRT-Fc was generated according to the manufacturer's instruction.

Briefly, the transfer vector containing the shTRT-Fc and a homologous recombination sequence, was cotransfected with a partial sequence of the Ad5 genome into QBI-293A cells. Upon recombination, the El gene is lost from the viral DNA. However, QBI-293A cells contain the Ad5 E1 gene inserted into the chromosome and infectious adenovirus is produced using E1 in trans. Cells are overlayed with agarose, and plaques are purified, amplified, and then screened by PCR of viral lysates. After screening, each positive clone is tested for expression of recombinant protein and viral production. Control recombinant adenoviral vectors, ad-hTRT (expressing the native hTRT) and ad-eGFP, were also generated.

### Example 19

### Efficient Transduction of Human Monocyte-Derived DCs by Adenoviral Vectors

Human monocyte-derived DCs were generated. Briefly, PBMCs isolated by Ficoll-Hypaque gradient centrifugation of buffy coats from healthy donors were washed three times in PBS and resuspended in RPMI-1640 with 10% FCS. The cells were allowed to adhere differentially in a volume of 25 ml (2-3×10⁶ cells/ml) to 150-cm² plastic tissue culture flasks for one hour at 37° C. in humidified 5% CO₂. The non-adherent cells were removed by rinsing three times with PBS. Remaining adherent cells were harvested and cultured at a density of 1×10⁶ cells/ml in complete RPMI medium. 800 U/ml human recombinant GM-CSF and 500 U/ml human rIL-4 (Biosource International) (R&D Systems) were added to the culture medium. At different times of culture, the cells were recovered by vigorous washing with 0.02% EDTA in PBS. Titrated virus stocks were thawed at 37° C., and transductions of DCs were performed using MOIs ranging from 1 to 1,000. Recombinant adenovirus was added to DC cultures on different days at an estimated MOI of 10-1,000 and the plates were centrifuged at 1,000×g for 90 minutes. Two days after transfection, eGFP expression in DCs was analyzed by flow cytometry and over 90% of DCs were eGFP positive when infected with adenoviral vectors at an MOI of 500 or higher.

### Example 20

### To Evaluate Anti-Tumor Activity of Retrogen-DCs in Mice

A TC-1 tumor cell line, which was generated by cotransforming primary lung cells of C57BL/6 mice with hTRT and activated ras oncogene, is used.

C57BL/6 mice (4-6 weeks old) in several groups are immunized with DCs transfected with shTRT-Fc or different control vectors by different routes (intradermal, subcutaneous, and intravenous) one to three times at two week intervals. Transfected DCs with or without TNF-maturation are used to evaluate anti-tumor activity. Two weeks after the last immunization, the mice are inoculated with the hTRT expressing tumor cells TC-1 (3-5×10⁵) by sc injection over the flank. Tumor development are observed and measured by caliper in three dimensions to determine the incidence of tumors, volumes, and growth curves of tumors that develop in different groups.

### Example 21

### Inhibition of Tumor Growth

C57BL/6 mice (6-8 each group) are inoculated with the hTRT-TC-1 cells (0.2-5×10⁶) by subcutaneous injection over the flank. After tumor growth, the mice are divided into different groups and immunized with DCs transfected with different constructs. Different administration routes, including intradermal, subcutaneous, and intravenous, are evaluated. Also, different numbers of DCs for each injection (0.1-5×10⁶) and different injection frequencies (1-3 times at two week intervals) are evaluated. After the treatment, tumor development are observed and measured by caliper in three dimensions to determine the tumor volumes, growth curves, and animal survival.

### Example 22

### Inhibition of Metastases

C57BL/6 mice are injected intravenously via the tail vein with 1-5×10⁵ TC-1 cells. One week later, the mice are treated with DCs transfected with different constructs. The DC injection route and dosage used are determined in the above study. Mice are killed at 14-22 days after tumor challenge. All lobes of both lungs are dissected, and surface lung metastases are scored and counted under a dissecting microscope.

### Example 23

### Anti-Tumor Activity of hTRT Retrogen-DCs Compared with hTRT Protein-Loaded or Ii-hTRT Transfected DCs in Mice

The method for protein-loading DCs is performed as described previously. Briefly, PBMC-derived DCs (5×10⁵/ml) are incubated at 37° C., 5% CO₂ for 18-20 h in complete medium supplemented with IL-4 (100 U/ml) and GM-CSF (100 ng/ml) in the presence of the recombinant hTRT protein (20 to 100 μg/ml). The protein-loaded DCs are then used to stimulate T-cells in vitro. The culture condition and re-stimulation schedule for priming T-cells by protein-loaded DCs follow those for the transfected DCs in order to compare their potency. The same numbers and administration routes of retrogen-DCs and hTRT protein-loaded DCs are used to evaluate anti-tumor activity, as described above.

Tumor development is observed and measured by caliper in three dimensions to determine the incidence of tumors, volumes, and growth curves of tumors that develop in different groups.

### Example 24

### Initial Identification of Promiscuous Class-II-Restricted Epitopes in hTRT

To identify promiscuous MHC class II Th epitopes in hTRT, the amino acid sequence of this protein was examined for the presence of peptide sequences containing binding motifs for multiple HLA-DR alleles using the algorithm program TEPITOPE (Hammer, 1997; Bono, 1999). The prediction threshold was set at 1% and peptides were selected on the basis of their ability to bind to at least three of the following eight HLA-DR molecules DRB1*0101, DRB1*0301, DRB1*0401, DRB1*0701, DRB1*0801, DRB1*1101, DRB1*1501, and DRB5*0101.

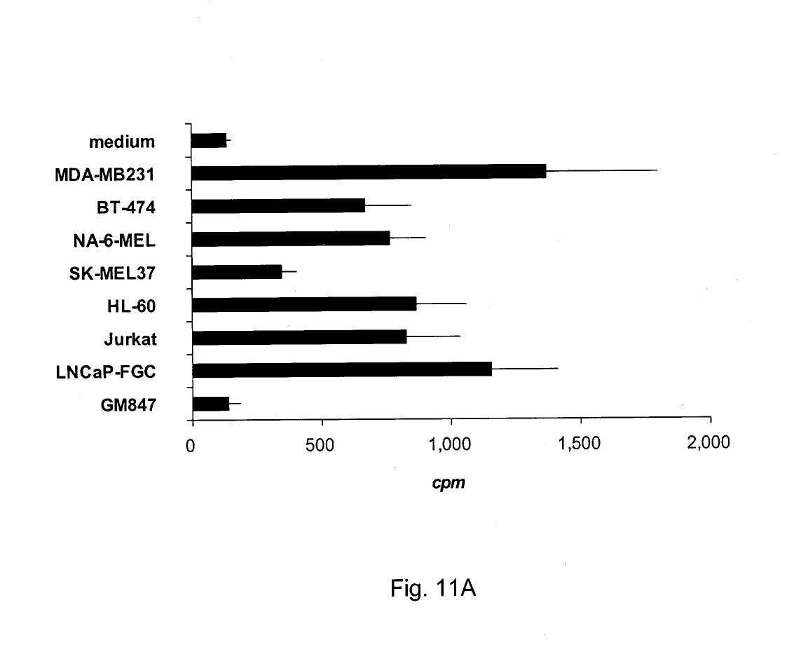

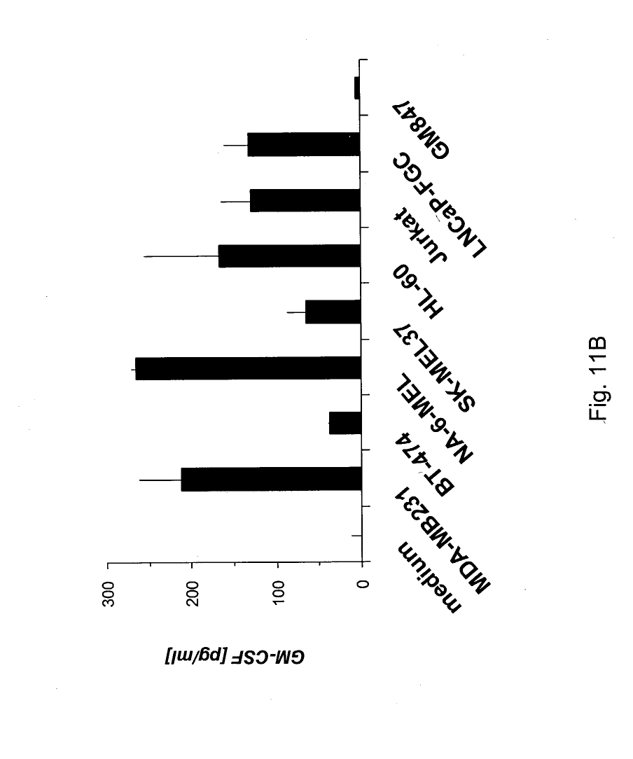

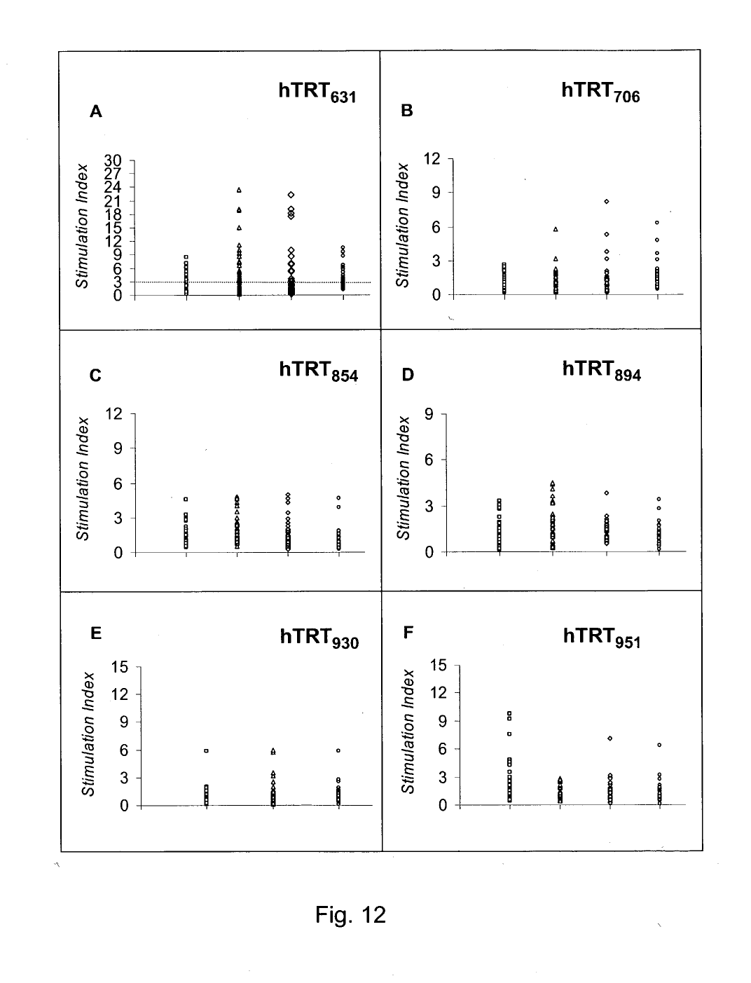

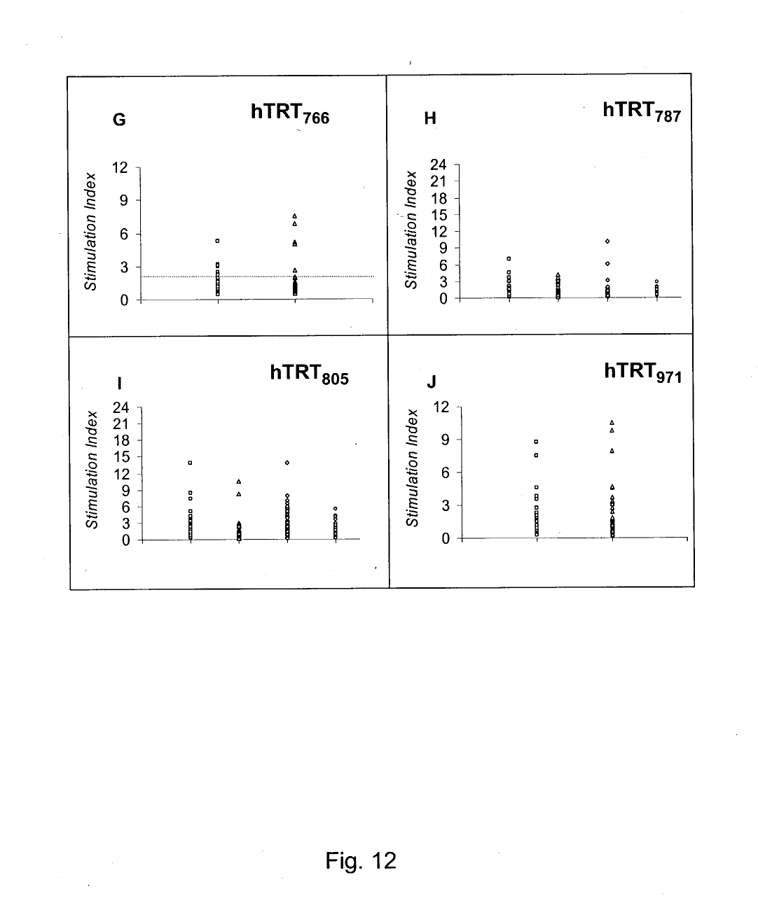

Because Th cells generally prefer to recognize peptides of about 15 residues in length, ten predicted peptides corresponding to promiscuous binding motifs of 15 mer or longer were synthesized and purified (Table 2). Human T-cell responses to these peptides were assessed by isolating PBMC from HLA-typed healthy donors with HLA-DR1, DR3, DR15, or other alleles and seeding them into 96-well plates that were subsequently stimulated with each peptide. After a week of stimulation, the cultures were tested for their capacity to respond to the peptides presented by autologous PBMC. Cultures exhibiting at least a 3-fold increase in their proliferative response to peptides were considered positive. Stimulation indexes (SI) representing PBMC responses to each of the 10 peptides were shown in FIG. 12A-FIG. 12J. Almost all donors tested responded to hTRT631, hTRT706, hTRT854, hTRT894, hTRT930, hTRT951, hTRT766, hTRT787, hTRT805, and hTRT971, indicating that the ten peptides were viable Th epitope candidates. Importantly, several peptides (hTRT631, hTRT894, hTRT766, hTRT787, and hTRT805) were capable of inducing T cell responses to more than one MHC class II allele, indicating some degree of promiscuity.

TABLE 2
hTRT Peptides

| | |
|---|---|
| RPIVNMDYVVGARTFRREKR | (SEQ.ID.NO.26, hTRT631) |
| LYFVKVDVTGAYDTI | (SEQ.ID.NO.89, hTRT706) |
| XLTDLQPYMRQFVAHL | (SEQ.ID.NO.59, hTRT766) |
| XRDAVVIEQSSSLNEA | (SEQ.ID.NO.62, hTRT787) |
| LFDVFLRFMCHHAVRIRGK | (SEQ.ID.NO.90, hTRT805) |
| FAGIRRDGLLLRLVD | (SEQ.ID.NO.91, hTRT854) |
| YGCVVNLRKTVVNFP | (SEQ.ID.NO.22, hTRT894) |
| WCGLLLDTRTLEVQS | (SEQ.ID.NO.92, hTRT930) |
| RTSIRASLTFNRGFK | (SEQ.ID.NO.93, hTRT951) |
| RRKLFGVLRLKCHSLFLDL | (SEQ.ID.NO.94, hTRT971) |

### Example 25

### Recombinant Protein, Monoclonal Antibodies, and Tissue Culture Reagents

Recombinant hTRTaa540-aa1003-Fc and Neu(extracellular domain)-Fc fusion proteins were produced in SF9 insect cells by use of a baculovirus expression system (Gibco, Grand Island, N.Y.), purified by affinity binding to Protein A (Sigma, St. Louis, Mo.), and tested by Western blot analysis with anti-hTRT (Santa Cruz Biotechnology, Santa Cruz, Calif.) or anti-Neu (Oncogene, La Jolla, Calif.) antibodies, respectively. The following hybridomas were used to produce monoclonal antibodies: HB55 (L243, anti-human HLA-DR, ATCC), HB95 (W6/32, anti-human MHC class I, ATCC), HB103 (Genox3.53, anti-human HLA-DQ, ATCC), HB180 (9.3F10, anti-human MHC class II, ATCC), and 2D6 (anti-human HLA-DR and HLA-DQ monomorphic). Anti-human CD4 (RPA-T4, FITC labeled), anti-human CD8 (HIT8a, PE labeled), anti-human CD4 (PE labeled), anti-human HLA-DR (FITC labeled) and anti-mouse CD4 (FITC labeled) were all purchased from BD PharMingen (San Diego, Calif.). Media used for cell culture were AIM-V serum-free medium (Life Technologies, Inc., Grand Island, N.Y.), RPMI 1640 supplemented with 10% FBS (Life Technologies, Inc., Grand Island, N.Y.) and L-glutamine/penicillin/streptomycin, and CellGenix DC serum-free medium (CellGenix, Germany). Human recombinant IL-2 was purchased from Boehringer Roche (Indianapolis, Ind.).

### Example 26

### Specificity and MHC Restriction Analysis of CD4+ T-cell Clones

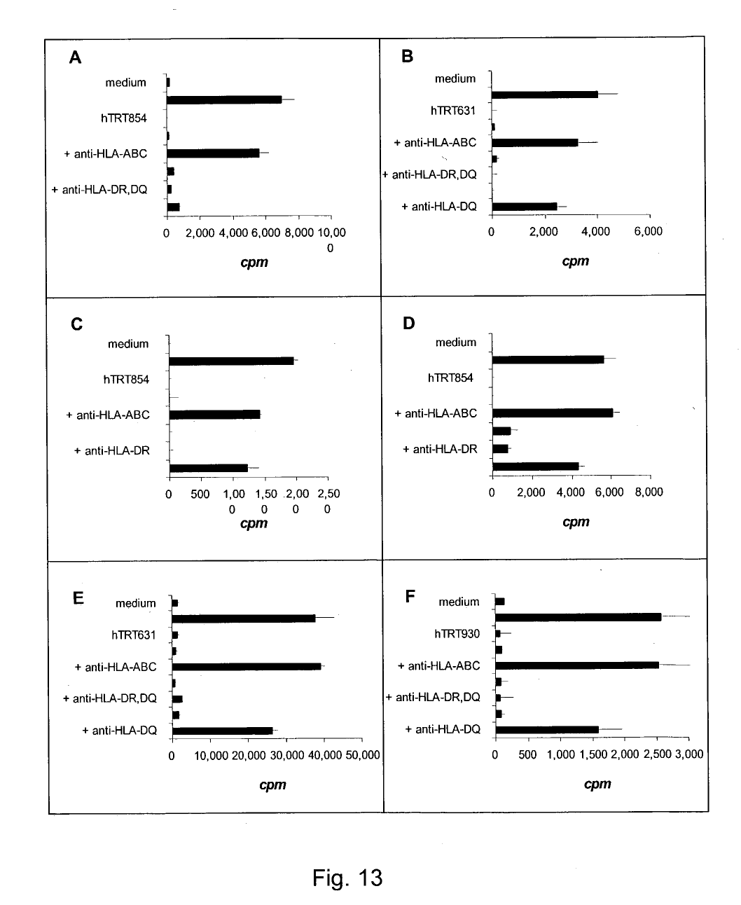

To further characterize the peptides that induced positive T-cell responses, cultures of T-cells were selected and expanded cultures that exhibited at least a 3-fold increase in their proliferative response to peptides and cloned them by limiting dilution. T-cell clones that proliferated in response to six peptides (hTRT631, hTRT706, hTRT766, hTRT787, hTRT805, and hTRT894) were successfully generated. Those specific for the remaining peptides (hTRT854, hTRT930, hTRT951, and hTRT971) failed to expand to sufficient numbers for further analysis despite repeated attempts with different donors' blood. FIG. 13A-FIG. 13F showed the specificity of the T-cell clone responses to the various peptides. All T-cell clones responded vigorously to the stimulation with the corresponding peptides, but did not respond to stimulation with irrelevant 15-mer peptides derived from EBNA1 or with irrelevant, non-corresponding peptides.

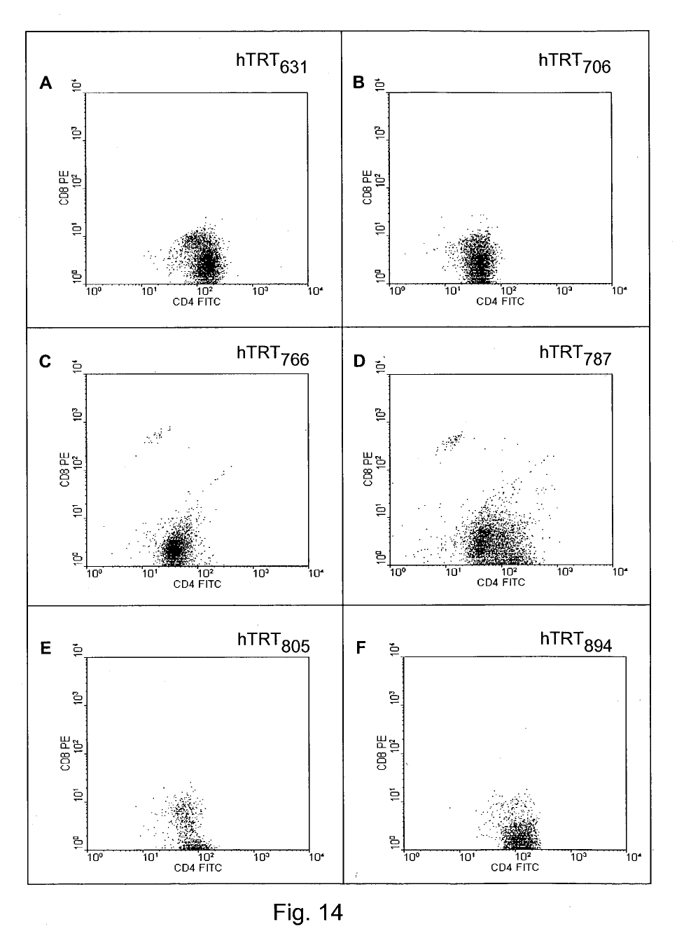

Further, antibody blocking assays were used to test whether the T-cell responses to peptides were MHC class II-restricted. As shown in FIG. 13A-FIG. 13F, responses of the hTRT631-, TRT706-, hTRT766-, hTRT787-, hTRT805-, and hTRT894-specific T-cell clones were all inhibited by anti-HLA-DR and anti-HLA-DR/DQ/DP antibodies, but not by anti-HLA-ABC (class I) and anti-HLA-DQ antibodies. Flow cytometric analysis of the responses to T-cell clones (FIG. 14A-FIG. 14F) confirmed clones were CD4-positive and CD 8-negative.

### Example 27

### Identification of Natively Processed Epitopes

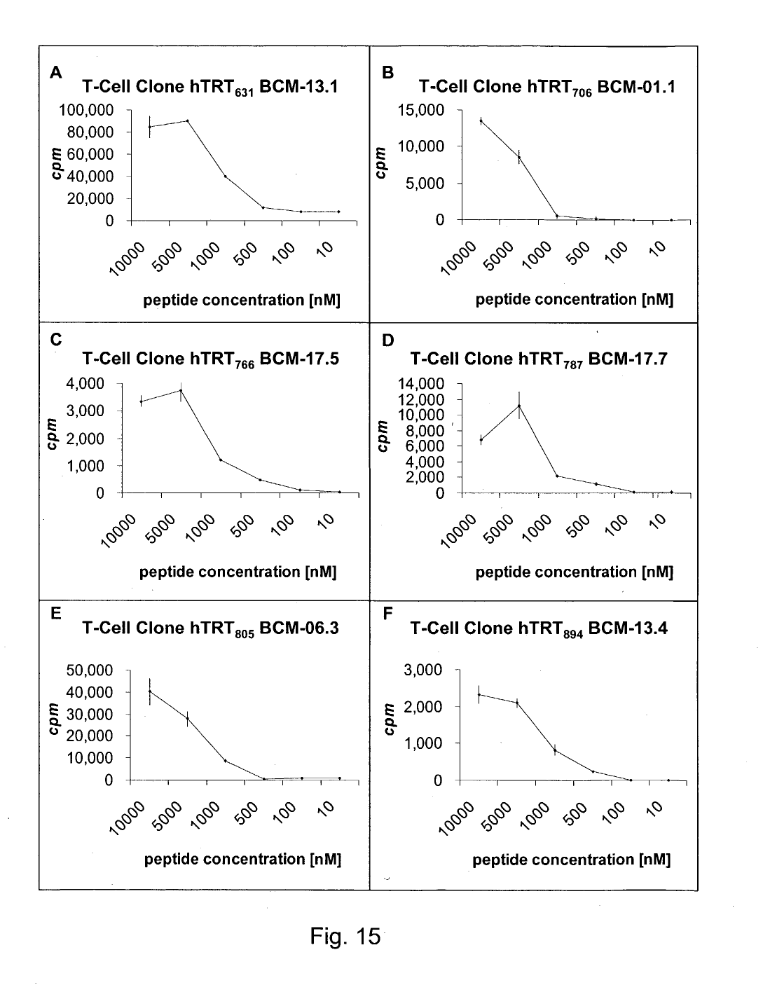

The effectiveness of antitumor immunotherapy based on CD8+ and CD4+ T-cells depends on the ability of the latter to recognize naturally processed antigen presented by APC. This property depends in turn on correct processing of the epitope in the MHC class II pathway and the avidity of the epitope for its MHC/TCR-complex (Kobayashi, 2000). To determine whether the newly identified peptides were naturally processed antigens, the avidity of the specific T-cell clones for their ligands was evaluated. Peptide titration curves were generated with autologous DC as APC. For peptides hTRT631, hTRT766, hTRT787, the peptide concentrations required to obtain half of the maximal proliferation exceeded 1.0 μM (FIG. 15A-FIG. 15F). For hTRT706, hTRT805, and hTRT894, half maximal proliferation was observed at higher concentrations (>5.0 μM).

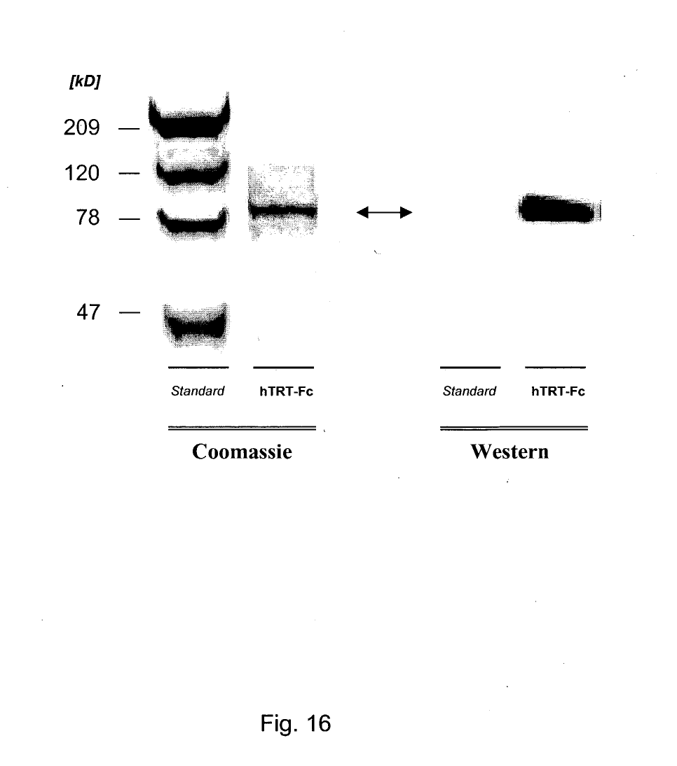

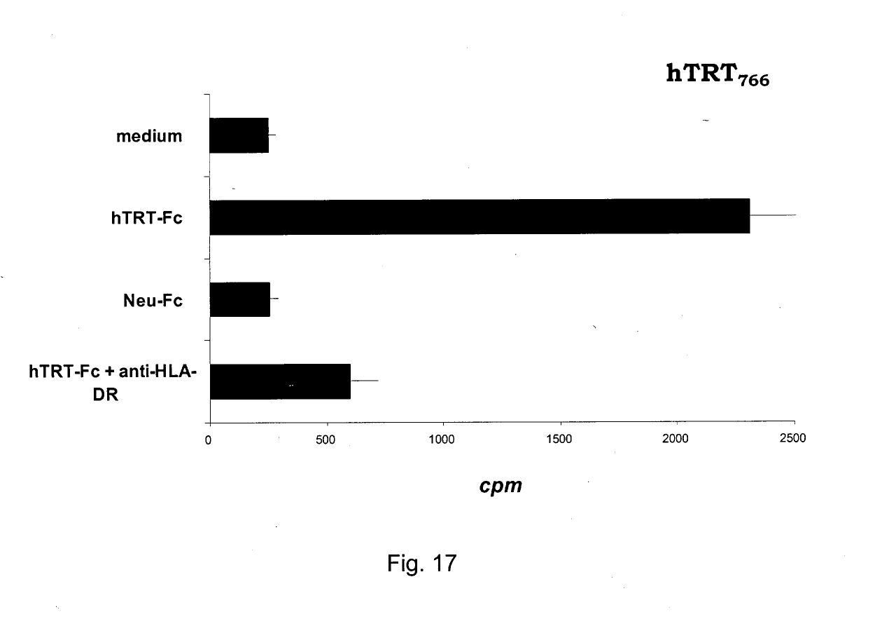

The ability of these T-cell clones to recognize naturally processed antigen in the form of recombinant hTRT protein was tested in experiments in which autologous PBMC or DC were used as APC and recombinant hTRT proteins as a source of antigen (FIG. 16). As shown in FIG. 17, the hTRT766-specific T-cell clone responded to hTRT protein-pulsed DC and this activity was inhibited by anti-HLA-DR antibody. The response of the hTRT766-specific T-cell clone to the hTRT protein was specific, since the T-cells did not react to stimulation with autologous DC pulsed with irrelevant recombinant Neu-Fc protein. Subsequent testing of T-cell clones specific for hTRT787 or hTRT631, which showed avidities similar to that of hTRT766-specific clones, failed to detect significant proliferative responses when either PBMC or DC were used as APC. Thus, hTRT631 and hTRT787 wer cryptic epitopes, not produced by APC that normally process protein antigen.

T-cell clones specific for hTRT706, hTRT805, or hTRT894- had lower avidities for their ligands and were unable to proliferate when stimulated with PBMC or DC pulsed with the corresponding hTRT protein. This result suggested either of two possibilities: either these epitopes were cryptic or the affinity of the T cells for the epitopes was low, requiring a higher number of peptide/MHC complexes than normally expressed on the APC to trigger proliferative T-cell responses. Taken together, the data indicated that of the 10 peptides tested only hTRT766 represented a naturally processed Th epitope in hTRT.

### Example 29

### Promiscuity of Naturally Processed hTRT Epitopes

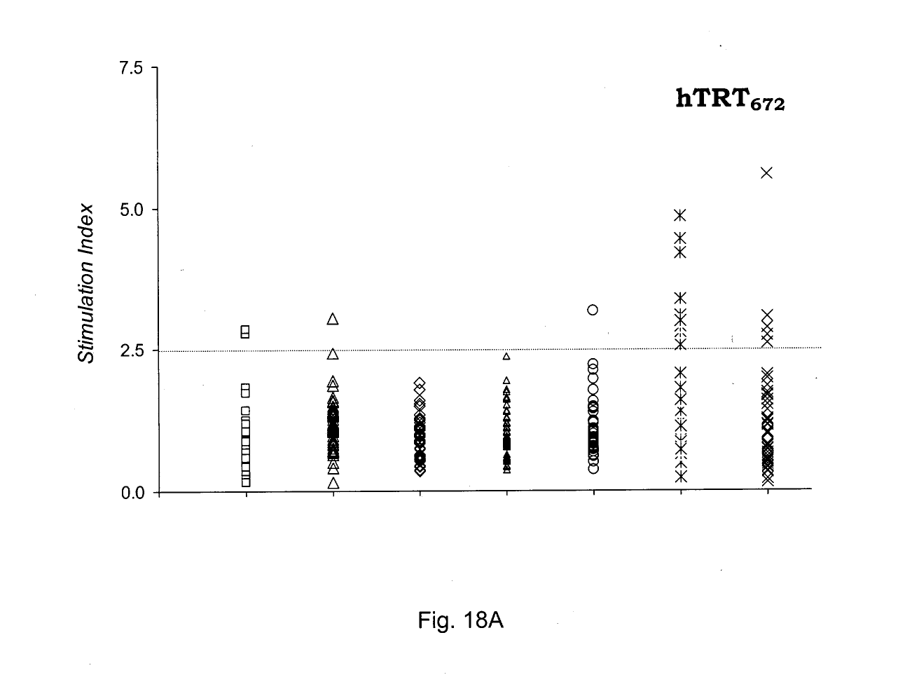

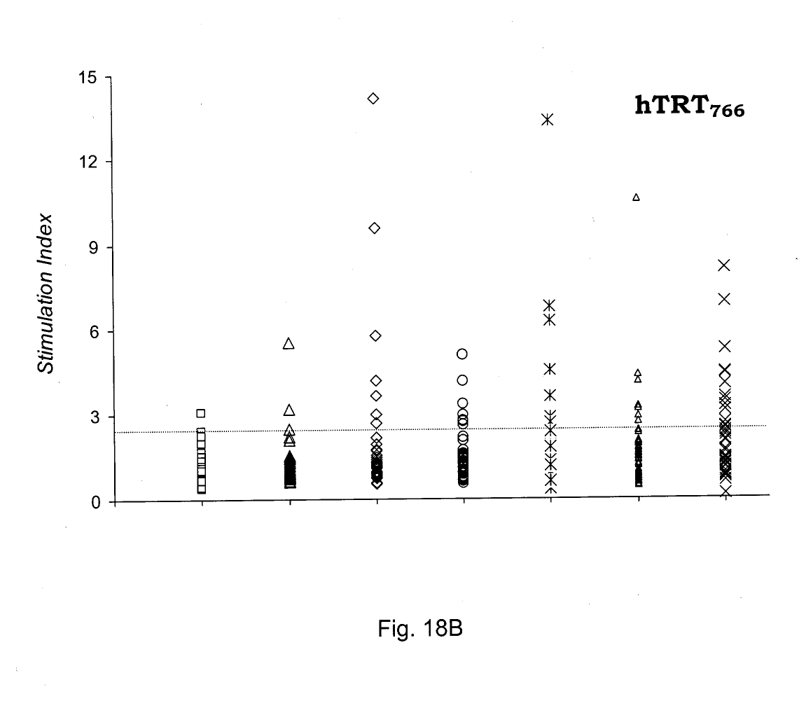

The promiscuity of the naturally processed epitope hTRT766 as well as the previously identified epitope hTRT672 (Schroers, 2000) was tested. PBMC from donors with different HLA-DR alleles were stimulated with either peptide for 1 week using autologous PBMC as APC. The cultures were then tested for their capacity to respond to the peptide presented by autologous PBMC. As shown in FIG. 18A and FIG. 18B, T-cells from donors with genotypes of DR01/11, DR04/04, DR07/07, DR04/08, DR15/16, and DR03/15 all responded to hTRT766, while T-cells from donors with genotypes of DR13/14 and DR15/16 responded to hTRT672. These results indicated that both naturally processed epitopes, especially hTRT766, served as promiscuous MHC class II Th epitopes capable of inducing CD4+ T-cell responses in the context of several HLA-DR alleles.

### Example 30

### T-Cell Precursor Frequencies in Healthy Donors and Cancer Subjects

Since hTRT is a self-antigen, hTRT-specific CD4+ T-cells are largely deleted during T-cell negative selection in the thymus. Thus, it is important to assess the frequency of hTRT-specific CD4+ T-cells in humans.

Briefly, the T-cell precursor frequency was calculated as the number of positive wells/total number of T-cells in all wells tested, since others have demonstrated that an antigen-specific T-cell line derived from a 96-plate well (200,000 cells/well) most likely originated from a single T-cell precursor (Zhang, 1994). The frequencies of T-cell precursors specific for the naturally processed epitopes hTRT766 and hTRT672 in different DR donors were 0.1-1.14×10⁶ and 0-0.83×10⁻⁶, respectively (Table 3). Interestingly, the frequencies of T-cell precursors specific for cryptic peptides not processed from native antigen and presented be APC appeared to be higher than results for hTRT766 and hTRT672.

TABLE 2

| | Number of wells with SI > 2.5/ | | Estimated Frequency of |
|---|---|---|---|
| Peptide | total number of tested wells | | hTRT-specific T-cells |
| HTRT₆₃₁ | Donor DR 3/11: | 9/48 | 0.9-2.8 × 10⁻⁶ |
| | Donor DR 3/15a: | 27/48 | |
| | Donor DR 14/15: | 16/48 | |
| | Donor DR 3/4: | 27/48 | |
| hTRT₆₇₂ | Donor DR 1/1: | 2/48 | 0-0.83 × 10⁻⁶ |
| | Donor DR 1/11: | 1/48 | |
| | Donor DR 4/4: | 0/48 | |
| | Donor DR 3/4: | 0/48 | |
| | Donor DR 7/7: | 1/48 | |
| | Donor DR 15/16: | 8/48 | |
| | Donor DR 13/14: | 4/48 | |
| hTRT₇₀₆ | Donor DR 3/15^{a}: | 1/48 | 0.1-0.41 × 10⁻⁶ |
| | Donor DR 3/15^{b}: | 2/48 | |
| | Donor DR 3/4: | 4/48 | |
| | Donor DR 3/11: | 4/48 | |
| hTRT₇₆₆ | Donor DR 4/8: | 5/48 | 0.1-1.14 × 10⁻⁶ |
| | Donor DR 1/11: | 5/48 | |
| | Donor DR 1/1: | 1/48 | |
| | Donor DR 4/4: | 7/48 | |
| | Donor DR 3/15: | 6/48 | |
| | Donor DR 7/7: | 7/48 | |
| | Donor DR 15/16: | 9/48 | |
| | Donor DR 4/8: | 11/48 | |
| HTRT₇₈₇ | Donor DR 3/15: | 4/48 | 0.1-0.52 × 10⁻⁶ |
| | Donor DR 3/4: | 5/48 | |
| | Donor DR 1/11: | 3/48 | |
| | Donor DR 4/7: | 1/48 | |
| HTRT₈₀₅ | Donor DR 14/15: | 11/48 | 0.41-2.29 × 10⁻⁶ |
| | Donor DR 1/1: | 4/48 | |
| | Donor DR 13/14: | 22/48 | |
| | Donor DR 7/7: | 6/48 | |
| HTRT₈₅₄ | Donor DR 3/11: | 6/48 | 0.20-0.62 × 10⁻⁶ |
| | Donor DR 1/3: | 8/48 | |
| | Donor DR 3/4: | 6/48 | |
| | Donor DR 3/15: | 2/48 | |
| hTRT₈₉₄ | Donor DR 3/15^{a}: | 7/48 | 0.1-0.73 × 10⁻⁶ |
| | Donor DR 14/15: | 7/48 | |
| | Donor DR 1/3: | 1/48 | |
| | Donor DR 3/15^{b}: | 2/48 | |
| hTRT₈₉₄ | Donor DR 3/15^{a}: | 1/48 | 0.1-0.31 × 10⁻⁶ |
| | Donor DR 3/4: | 3/48 | |
| | Donor DR 3/15^{b}: | 3/48 | |
| hTRT₈₉₄ | Donor DR 3/15^{a}: | 10/48 | 0.31-1.0 × 10⁻⁶ |
| | Donor DR 1/3: | 3/48 | |
| | Donor DR 3/4: | 4/48 | |
| | Donor DR 3/15^{b}: | 3/48 | |

Next, T-cell responses against hTRT766 and hTRT672 were induced using PBMC from cancer subjects. Due to the limited amount availability of subject blood, testing was restricted to a single naturally processed epitope, hTRT672, and one cryptic epitope hTRT631. As shown in Table 4, out of 7 prostate cancer subjects tested, T-cells from 3 subjects with different HLA DR alleles responded to the hTRT672 stimulation, further demonstrating the promiscuity of the hTRT672 epitope. The precursor frequencies of T-cells specific for the epitope hTRT672 in the positive donors with different DR types are 0 to 0.41×10⁻⁶ (Table 4). Similar to the results obtained with healthy donors, the frequencies of T-cell precursors specific for the naturally processed epitope appeared to be lower than those for the cryptic hTRT631 peptide (Table 4). Thus, the data demonstrated that CD4+ T-cell precursors specific for the two naturally processed epitopes, hTRT766 and hTRT672, were part of normal human T-cell repertoires and, when properly stimulated, were readily activated in healthy donors and prostate cancer subjects.

TABLE 3

| | | | Positive wells/ | Estimated |
|---|---|---|---|---|
| Peptide | Donor | HLA | total wells | Frequency |
| hTRT₆₃₁ | PCa 01 | DR 4/11 | 6/48 | 0.63 × 10⁻⁶ |
| | PCa 02 | DR 4/13 | 2/48 | 0.21 × 10⁻⁶ |
| | PCa 03 | DR 3/3 | 7/48 | 0.73 × 10⁻⁶ |
| | PCa 04 | DR 11/13 | 13/48 | 1.35 × 10⁻⁶ |
| | PCa 08 | DR 7/11 | 2/48 | 0.21 × 10⁻⁶ |
| | PCa 11 | DR 4/11 | 3/24 | 0.63 × 10⁻⁶ |
| hTRT₆₇₂ | PCa 03 | DR 3/3 | 0/48 | 0 |
| | PCa 04 | DR 11/13 | 4/48 | 0.41 × 10⁻⁶ |
| | PCa 05 | DR 7/7 | 0/48 | 0 |
| | PCa 06 | DR 7/11 | 3/48 | 0.31 × 10⁻⁶ |
| | PCa 07 | DR 7/8 | 1/48 | 0.10 × 10⁻⁶ |
| | PCa 08 | DR 7/11 | 1/48 | 0.10 × 10⁻⁶ |
| | PCa 09 | DR 4/13 | 1/48 | 0.10 × 10⁻⁶ |
| | PCa 11 | DR 4/11 | 1/48 | 0.10 × 10⁻⁶ |
| | PCa 12 | DR 7/13 | 0/48 | 0 |
| | PCa 13 | DR 4/15 | 4/48 | 0.41 × 10⁻⁶ |

### Example 31

### Evaluation of T Cell Responses by IFN-γ ELISPOT Assay

IFN-γ ELISPOT assay was used to analyze peptide-specific T cell responses by determining the frequency of Th precursors specific for the peptide. Mice were sacrificed 14 days after the last immunization and splenocytes were obtained for assessing IFN-γ production.

Briefly, 96-well MultiScreen-IP plates (Millipore Corporation, Bedford, Mass.) were coated with 100 ul/well capture mAb against mouse IFN-γ (AN-18, Mabtech Inc, Cincinnati, Ohio) at a concentration of 10 μg/ml and incubated overnight at 4° C. The plates were washed 4 times with PBS, then blocked with RPMI 1640 plus 10% FBS for 2 hours at 37° C. After washing, freshly isolated splenocytes were plated at 2×10⁵cells/well in RPMI 1640 with 10% FBS, in the presence or absence of peptide hTRT766 (20 μg/ml), recombinant hTRT proteins (20 μg/ml) or hTRT-positive NA-6-Mel (ATCC) tumor lysates (50 ul/well). Tumor cell lysates were prepared by three freeze-thaw cycles of 5×10⁷ tumor cells resuspended in 5 ml of RPMI 1640 with 10% FBS. Then the cells were centrifuged at 15,000 g for 30 minutes at 4° C. Supernatant was recovered, aliquoted and stored at −80° C. for later use, as described previously [Schroers, 2002 #116]. After 20 hours of cell culture in the incubator, the cells were removed by washing 3 times with PBS and 4 times with PBS/Tween20 (0.05%). Biotinylated anti-mouse IFN-γ antibody (R4-6A2, Mabtech Inc, Cincinnati, Ohio), diluted to 1 μg/ml in PBS/Tween20 containing 0.5% bovine serum albumin, was added and incubated for 2 hours at 37° C. The plates were then washed 6 times with PBS/Tween20 (0.05%) and subsequently avidin-peroxidase-complex (Vector Laboratories, Burlingame, Calif.) was added and incubated for 1 hour at room temperature, and removed by washing 3 times with PBS and PBS/Tween20 (0.05%). The color of the plates was developed by adding HRP substrate 3-amino-9-ethylcarbozole (Sigma, St. Louis, Mo.). The plates were then washed with tap water, and air dried in dark. The plates were evaluated using an automated ELISPOT reader (Zellnet Consulting Inc, New York, N.Y.).

### Example 30

### Antigen-Specific T Cell Response Induced by hTRT766 Immunization of HLA-DR Transgenic Mice

To further assess the therapeutic potential of hTRT766, HLA-DR4 transgenic mice (Congia, 1998; Sonderstrup, 1998; Sonderstrup, 1999) were used to determine whether immunization with the peptide induces a CD4+ Th response specific not only for the peptide, but also for the hTRT protein.

Briefly, human HLA DR4 transgenic mice (HLA-DRB1*0401), which are murine class II-deficient and transduced with human CD4 molecule, were generated. [Congia, 1998; Sonderstrup, 1998; Sonderstrup, 1999). The transgenic mice were successfully used to identify human class-II-restricted epitopes and to study immune responses (Congia, 1998; Sonderstrup, 1998; Sonderstrup, 1999; Geluk, 1998). HLA DR4 expression on the transgenic mice was analyzed by flow cytometry. Male DR4 transgenic mice 6- to 10-week-old were used for experiment. The transgenic mice were immunized twice at one week interval with 100 μg of hTRT766 peptide emulsified in complete Freunds adjuvant (CFA) (final volume, 100 μl) and administered subcutaneously (s.c.) into the rear back. Control group mice were injected with phosphate-buffered saline (PBS) emulsified in CFA.

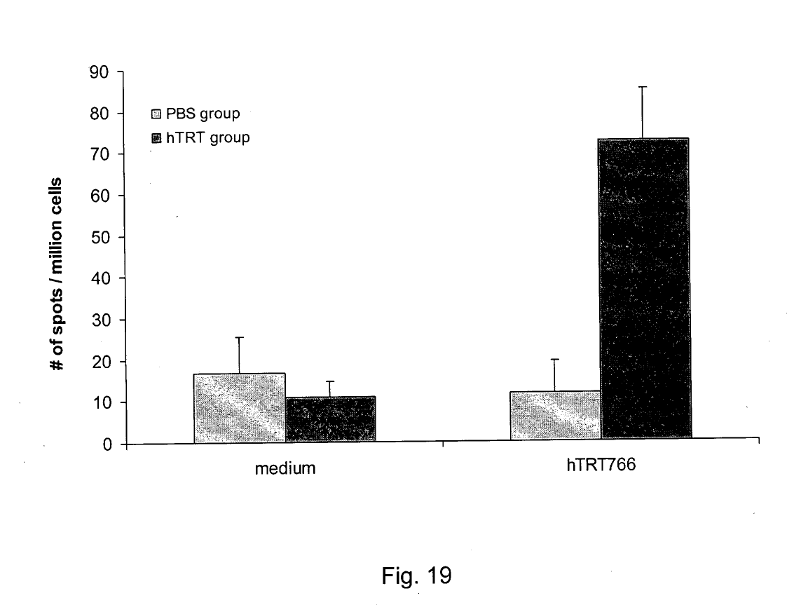

The spleen cells were isolated and stained with FITC-conjugated mouse anti-human HLA-DR, PE-conjugated mouse anti-human CD4 or FITC-conjugated rat anti-mouse CD4 (BD PharMingen, San Diego, Calif.). Flow cytometric analysis determined that these transgenic mice were human HLA-DR, CD4 positive and mouse CD4 negative. Ten days after the last immunization, the transgenic mice were sacrificed and the responses of their splenocytes to peptides, recombinant hTRT protein and hTRT-positive tumor cell were examined by using IFN-γ ELISPOT assays. The splenocytes of hTRT766-immunized mice responded strongly to the hTRT766 stimulation, producing IFN-γ at a frequency of 72 spots per million of splenocytes (medium control, 10/106). In contrast, the splenocytes of PBS-immunized control mice produced IFN-γ at a background frequency of 12 spots per million splenocytes to the peptide hTRT766 (medium control, 16/106) (FIG. 19). The splenocytes of hTRT766-immunized mice did not respond to an irrelevant peptide hTRT854 stimulation, indicating that T-cells induced by peptide immunization specifically responded to the immunized peptide.

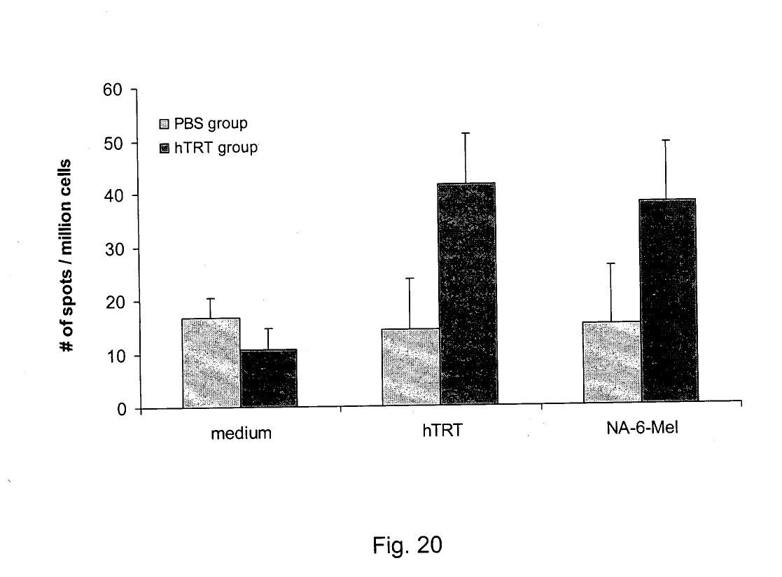

Since most of tumor cells are MHC class-II negative, the tumor-specific MHC class II-restricted CD4+T cells are not able to recognize these tumor cells directly. CD4+ T-cells induced by peptide immunization can react with antigen presenting cells (APCs) that take up and process the tumor antigen protein. Thus, transgenic mouse T-cells were tested to determined of they were activated when co-cultured with splenocytes containing T-cells and APCs pulsed with the recombinant hTRT proteins. As shown in FIG. 20, when stimulated with the recombinant hTRT protein, the splenocytes of hTRT766-immunized mice produced IFN-γ at a frequency of 41 spots per million cells (medium control, 10/106), significantly higher than the splenocytes of the PBS-immunized mice (frequency of 14 spots per million cells. Furthermore, the splenocytes of hTRT766-immunized mice produced IFN-γ at a background frequency, when stimulated with irrelevant CEA-Fc proteins (10/106). These results indicated that hTRT766 immunization activated T-cells that recognized antigenic peptides processed from hTRT proteins.

Finally, activated CD4+ T-cells were tested to determine if they recognize APCs that directly take up and process the tumor antigen from tumor cells. Melanoma cell line (NA-6-Mel) that expresses hTRT (Schroers, 2002) was used for this assay. As shown in FIG. 20, when stimulated with NA-6-Mel cell lysates, the splenocytes of hTRT766-immunized mice produced IFN-γ at a frequency of 38 spots per million cells (medium control, 10/10⁶), significantly higher than the splenocytes of the PBS-immunized mice at a frequency of 15 spots per million cells. The splenocytes of hTRT766-immunized mice produced IFN-γ at a background frequency, when stimulated with hTRT-negative GM847 (Schroers, 2002) tumor lysates (10/106), suggesting that T-cells activated by hTRT766 immunization specifically responded to antigenic peptides derived from hTRT-positive tumor.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Abbas et al., Nature. 383: 787-93, 1996.

Ausubel et al., (1997), Current Protocols in Molecular Biology, John Wiley & Sons, New York.

Baichwal and Sugden, In: Gene Transfer, Kucherlapati R, ed., New York, Plenum Press, pp. 117-148, 1986.

Bennett et al, Journal of Experimental Medicine. 186: 65-70, 1997.

Bennett et al., Nature 393, 478-80, 1998.

Blackburn, Annu Rev Biochem 61, 113-29, 1992.

Chaux et al., J Exp Med 189, 767-78, 1999.

Chen and Okayama, Mol. Cell Biol., 7:2745-2752, 1987.

Chen et al., Nature Medicine 3, 1110-6, 1997.

Cotton et al., Proc. Nat'l Acad. Sci. U.S.A. 89:6094-6098, 1992.

Coupar et al., Gene, 68:1-10, 1988.

Curiel, Ann, N.Y. Acad. Sci. 716: 36-58, 1994.

Engelhard et al., Annual Review of Immunology. 12: 181-207, 1994.

Fechheimer et al., Proc. Nat'l Acad. Sci. USA, 84:8463-8467, 1987.

Fraley et al., Proc. Nat'l Acad. Sci. USA, 76:3348-3352, 1979.

Friedmann, Science, 244:1275-1281, 1989.

Geijtenbeek et al., Cell 100, 575-85, 2000.

Ghosh and Bachhawat, “Targeting of liposomes to hepatocytes,” In: Liver Diseases, Targeted Diagnosis and Therapy Using Specific Receptors and Ligands,” Wu G. and C. Wu ed. New York: Marcel Dekker, pp. 87-104, 1991.

Gilboa, Immunity 11, 263-70, 1999.

Geluk et al., Proc Natl Acad Sci USA., 95:10797-802, 1998.

Gopal, Mol. Cell Biol., 5:1188-1190, 1985.

Graham and Van Der Eb, Virology, 52:456-467, 1973.

Grunhaus and Horwitz, Seminar in Virology, 3:237-252, 1992.

Hammer et al., Advances in Immunology. 66: 67-100, 1997.

Harland and Weintraub, J. Cell Biol., 101:1094-1099, 1985.

Hiyama et al., Journal of Immunology. 155: 3711-5, 1995.

Horwich et al., J. Virol., 64:642-650, 1990.

Hu et al., Journal of Experimental Medicine. 177: 1681-90, 1993.

Hung et al., Journal of Experimental Medicine. 188: 2357-68, 1998.

Inouye et al., J. Biol. Chem. 260: 7206-7213, 1985.

Jonuleit et al., European Journal of Immunology. 27: 3135-42, 1997.

Kaneda et al., Science, 243:375-378, 1989.

Kato et al., J. Biol. Chem., 266:3361-3364, 1991.

Kelleher and Vos, Biotechniques, 17:1110-1117, 1994.

Kim et al., Science. 266: 2011-5, 1994.

Klein et al., Nature, 327:70-73, 1987.

Kovacsovics-Bankowski et al., Science 267: 243-246, 1995.

Laughlin et al., J. Virol., 60:515-524, 1986.

Lebkowski et al., Mol. Cell. Biol., 8:3988-3996, 1988.

Macejak and Sarnow, Nature 35: 90-94, 1991.

Manici et al., J Exp Med 189, 871-6, 1999.

Mann et al., Cell, 33:153-159, 1983.

McLaughlin et al., J. Virol., 62:1963-1973, 1988.

Meyerson et al., Cell. 90: 785-95, 1997.

Minev et al., Proc Natl Acad Sci USA 97, 4796-801, 2000.

Morgan et al., Journal of Immunology. 160: 643-51, 1998.

Mumberg et al., Proc. Nat'l Acad. Sci. U.S.A 96: 8633-8,1999.

Muzyczka, Curr. Top. Microbiol. Immunol., 158:97-129, 1992.

Nair et al., Nature Medicine. 6: 1011-7, 2000.

Nakamura et al., Science. 277: 955-9, 1997.

Naldini et al., Proc. Nat'l Acad. Sci. U.S.A., 93: 11382-11388, 1996.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Ossendorp et al., Journal of Experimental Medicine. 187: 693-702, 1998.

Overwijk et al., Proc. Nat'l Acad. Sci. U.S.A. 96: 2982-7, 1999.

Pardoll and Topalian, Curr Opin Immunol 10, 588-94, 1998.

Paskind et al., Virology, 67:242-248, 1975.

Pelletin and Sonenberg, Nature 335: 320-325, 1998.

Perales et al., Proc. Nat'l Acad. Sci. 91:4086-4090, 1994.

Potter et al., Proc. Nat'l Acad. Sci. USA, 81:7161-7165, 1984.

Ramakrishnan et al., Cancer Research. 58: 622-5, 1998.

Ravetch, Cell 78, 553-60 (1994)

Remington's Pharmaceutical Sciences 15^{th} Edition, pg. 1035-1038 and 1570-1580.

Ridge et al., Nature. 393: 474-8, 1998.

Ridgeway, In: Vectors: A survey of molecular cloning vectors and their uses, Rodriguez R L, Denhardt D T, ed., Stoneham:Butterworth, pp. 467-492, 1988.

Rippe et al., Mol. Cell Biol., 10:689-695, 1990.

Rosenberg, Immunity 10, 281-7 (1999).

Rosenberg, Nature. 411: 380-4, 2001.

Sambrook et al., (1989) Molecular Cloning: A Laboratory Manual, Cold Springs Lab., New York.

Schoenberger et al., Nature 393, 480-3, 1998.

Schroers et al., Molecular Therapy: the Journal of the American Society of Gene Therapy. 1: 171-9, 2000.

Schroers et al., Cancer Res. 62:2600-5, 2002.

Shay and Bacchetti, European Journal of Cancer. 33: 787-91, 1997.

Sommerfeld et al., Cancer Research. 56: 218-22, 1996.

Sonderstrup et al., Immunol Rev. 164:129-38, 1998.

Sonderstup et al., Immunol Rev. 172:335-43, 1999.

Steinman, Cell 100, 491-4 (2000).

Temin, In: Gene Transfer, Kucherlapati (ed.), New York: Plenum Press, pp. 149-188, 1986.

Topalian et al., Proc Natl Acad Sci USA 91, 9461-5, 1994.

Tratschin et al., Mol. Cell. Biol., 4:2072-2081, 1984.

Tur-Kaspa et al., Mol. Cell Biol., 6:716-718, 1986.

van der Bruggen, et al., Science 254, 1643-7 (1991).

Vonderheide et al., Immunity. 10: 673-9, 1999.

Wagner et al., Proc. Nat'l Acad. Sci. 87:3410-3414, 1990.

Wang et al., Science 284, 1351-4 (1999).

Watts, Annu Rev Immunol 15, 821-50 (1997).

Weng et al., Journal of Experimental Medicine. 183: 2471-9, 1996.

Wong et al., Gene, 10:87-94, 1980.

Wu and Wu, J. Biol. Chem., 262:4429-4432, 1987.

You et al., Cancer Res 61, 197-205, 2001.

You et al., Immunol. 165, 4581-92, 2000.

Zhang et al., J Exp Med 179, 973-84, 1994.

Zhou and Tedder. Proc Natl Acad Sci USA 93, 2588-92, 1996.

Zufferey et al., Nat. Biotechnol., 15: 871-875, 1997.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein can be utilized according to the present invention. Accordingly, the appended claims are intended to include within their scope such processes, machines, manufacture, compositions of matter, means, methods, or steps.

What is claimed is:

1. An isolated polynucleotide sequence comprising the nucleic acid sequence of SEQ.ID.NO. 1.

2. An isolated polynucleotide sequence comprising the nucleic acid sequence of SEQ.ID.NO.2.

3. An isolated polypeptide comprising the amino acid sequence of SEQ.ID.NO.3.

4. The polypeptide of claim 3, wherein said amino acid sequence comprises epitopes that binds to MHC-I and MHC-II.

5. An isolated polypeptide comprising the amino acid sequence of SEQ.ID.NO.4.

6. The polypeptide of claim 5, wherein said amino acid sequence comprises epitopes that binds to MHC-I and MHC-II.

7. An isolated polypeptide comprising the amino acid sequence of SEQ.ID.NO.59.

8. The polypeptide of claim 7, wherein said amino acid sequence comprises epitopes that binds to MHC-II.

9. An expression vector comprising a nucleic acid sequence of SEQ.ID.NO.1.

10. An expression vector comprising a nucleic acid sequence of SEQ.ID.NO.2.

11. An expression vector comprising a polynucleotide encoding signal sequence, a polynucleotide encoding at least one epitope of human telomerase reverse transcriptase (hTRT), a polynucleotide encoding a cell binding element and a polynucleotide encoding a dendritic cell receptor, all operatively linked.

12. The expression vector of claim 11, wherein said epitope induces a CD4+ T-cell response in a mammal.

13. The expression vector of claim 11, wherein said epitope induces a CD4+ T-cell response and a CD8+T-cell response in a mammal.

14. The expression vector of claim 11, wherein the epitope of hTRT is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100.

15. An expression vector comprising a polynucleotide encoding signal sequence, a first polynucleotide sequence encoding at least one epitope of hTRT, a second sequence polynucleotide encoding at least one epitope of hTRT, a polynucleotide sequence encoding a cell binding element and a polynucleotide sequence encoding a dendritic cell receptor, all operatively linked.

16. The expression vector of claim 15, wherein the first and second polynucleotide sequences encoding at least one epitope of hTRT are separated by an internal ribosome entry site.

17. The expression vector of claim 15, wherein the first and second polynucleotide sequences encoding at least one epitope of hTRT are in tandem and under the control of one promoter.

18. The expression vector of claim 15, wherein the first polynucleotide sequences encoding at least one epitope of hTRT encodes an epitope that binds to a MHC-II receptor.

19. The expression vector of claim 18, wherein the second polynucleotide sequence encoding at least one epitope of hTRT encodes an epitope that binds to a MHC-II receptor.

20. The expression vector of claim 18, wherein the second polynucleotide sequence encoding at least one epitope of hTRT encodes an epitope that binds to a MHC-I receptor.

21. The expression vector of claim 15, wherein the first polynucleotide sequence encoding at least one epitope of hTRT encodes an epitope that binds to a MHC-I receptor.

22. The expression vector of claim 21, wherein the second polynucleotide sequence encoding at least one epitope of hTRT encodes an epitope that binds to a MHC-II receptor.

23. The expression vector of claim 15, wherein the polynucleotide sequence is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO. 13, SEQ.ID.NO.14, SEQ.ID.NO. 15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100.

24. An expression vector comprising a two transgenes, wherein the first and second transgene comprises a promoter polynucleotide sequence, a polynucleotide encoding signal sequence, a polynucleotide sequence encoding at least one epitope of hTRT, a polynucleotide sequence encoding a cell binding element, and a polynucleotide sequence encoding a dendritic cell receptor, all operatively linked.

25. The vector of claim 24, wherein the promoter polynucleotide sequence is the same for the first transgene and second transgene.

26. The vector of claim 24, wherein the promoter polynucleotide sequence is different for the first transgene and second transgene.

27. The vector of claim 24, wherein the polynucleotide sequence is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100.

28. A transformed cell comprising the expression vector of claim 11.

29. A transformed cell-comprising the expression vector of claim 15.

30. A transformed cell comprising the expression vector of claim 24.

31. A method of eliciting an immune response directed against an antigen, comprising the step of administering to a subject the expression vector of claim 9, 10, 11, 15, or 24.

32. A method of eliciting an immune response directed against an antigen comprising the step of administering to a patient a peptide selected from the group consisting of SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62,SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

33. A method of eliciting an immune response directed against an antigen comprising the step of administering to a subject a composition comprising SEQ.ID.NO.3 or SEQ.ID.NO.4.

34. A method of eliciting an immune response directed against an antigen comprising the step of administering to a subject the transformed cell of claim 28, 29, or 30.

35. A method of eliciting an immune response directed against an antigen comprising the step of administering to a subject cell lysate from the transformed cell of claim 28, 29, or 30.

36. A method of treating a hyperproliferative disease comprising the step of administering transduced antigen presenting cells to a subject via a parenteral route.

37. The method of claim 36, wherein hyperproliferative disease is further defined as cancer.

38. The method of claim 37, wherein said cancer is selected from the group consisting of lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

39. The method of claim 36, wherein hyperproliferative disease is further defined as immune-mediated.

40. The method of claim 36, wherein said antigen presenting cells are autologous to said subject.

41. The method of claim 36, wherein said antigen presenting cells are allogeneic to said subject.

42. The method of claim 36, wherein said antigen presenting cells are pulsed with an expression vector comprising a polynucleotide sequence of hTRT, wherein said polynucleotide sequence of is selected from the group consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.14, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99,and SEQ.ID.NO.100.

43. The method of claim 36, wherein said antigen presenting cells are pulsed with a peptide selected from the group consisting of SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62, SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

44. The method of claim 43, wherein the peptide is SEQ.ID.NO.59.

45. A method of treating a hyperproliferative disease comprising the step of administering to a subject an expression vector with a pharmaceutical acceptable carrier, wherein said expression vector comprises a polynucleotide promoter sequence, a polynucleotide encoding a signal sequence, a polynucleotide encoding an at least one epitope of hTRT, and a polynucleotide encoding a cell binding element and a polynucleotide sequence encoding a dendritic cell receptor, all operatively linked.

46. The method of claim 45, wherein the epitope of hTRT is selected from the group of polynucleotide sequences consisting of SEQ.ID.NO.1, SEQ.ID.NO.2, SEQ.ID.NO.5, SEQ.ID.NO.6, SEQ.ID.NO.7, SEQ.ID.NO.8, SEQ.ID.NO.9, SEQ.ID.NO.10, SEQ.ID.NO.11, SEQ.ID.NO.12, SEQ.ID.NO.13, SEQ.ID.NO.142, SEQ.ID.NO.15, SEQ.ID.NO.16, SEQ.ID.NO.95, SEQ.ID.NO.96, SEQ.ID.NO.97, SEQ.ID.NO.98, SEQ.ID.NO.99 and SEQ.ID.NO.100.

47. A method of treating a hyperproliferative disease comprising administering to a subject a hTRT specific peptide with a pharmaceutical acceptable carrier, wherein said peptide binds to a MHC-II receptor.

48. The method of claim 47, wherein said hTRT peptide is selected from the group of consisting of SEQ.ID.NO. 3, SEQ.ID.NO. 4, SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62,SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

49. The method of claim 48, wherein the peptide is SEQ.ID.NO.59.

50. A method of treating a hyperproliferative disease comprising administering to a subject a hTRT specific peptide with a pharmaceutical acceptable carrier, wherein said peptide binds to a MHC-I and MHC-II receptor.

51. The method of claim 50, wherein said hTRT peptide is selected from the group of consisting of SEQ.ID.NO. 3, SEQ.ID.NO. 4, SEQ.ID.NO.17, SEQ.ID.NO.18, SEQ.ID.NO.19, SEQ.ID.NO.20, SEQ.ID.NO.21, SEQ.ID.NO.22, SEQ.ID.NO.23, SEQ.ID.NO.24, SEQ.ID.NO.25, SEQ.ID.NO.26, SEQ.ID.NO.27, SEQ.ID.NO.59, SEQ.ID.NO.62,SEQ.ID.NO.77, SEQ.ID.NO.89, SEQ.ID.NO.90, SEQ.ID.NO.91, SEQ.ID.NO.92, SEQ.ID.NO.93 and SEQ.ID.NO.94.

52. The method of claim 51, wherein the peptide is SEQ.ID.NO.59.

53. A method of treating a hyperproliferative disease comprising the step of administering to a subject the expression vector of claim 9, 10, 11, 15, or 24.

54. A method of treating a hyperproliferative disease comprising the step of administering to a subject the transformed of claim 28, 29, or 30.

55. A method of treating a hyperproliferative disease comprising administering comprising the step of administering to a subject cell lysate of the transformed of claim 28, 29, or 30.
